(19) United States
(12) Patent Application Publication
(10) Pub. No.: US 2025/0114000 A1
(43) Pub. Date: Apr. 10, 2025

(54) SPECTROPOLARIMETRIC IMAGING OF THE EYE FOR DISEASE DIAGNOSIS

(71) Applicant: RETISPEC INC., Toronto (CA)

(72) Inventors: Eliav Shaked, Toronto (CA); Alon Hazan, Toronto (CA); Tommaso Alterini, Toronto (CA); Joni Petteri Teikari, Toronto (CA)

(73) Assignee: RETISPEC INC., Toronto (CA)

(21) Appl. No.: 18/918,794

(22) Filed: Oct. 17, 2024

Related U.S. Application Data
(63) Continuation of application No. PCT/IB2023/000209, filed on Apr. 20, 2023.
(60) Provisional application No. 63/425,155, filed on Nov. 14, 2022.
(60) Provisional application No. 63/332,855, filed on Apr. 20, 2022.

(51) Int. Cl. A61B 3/12; A61B 3/00; G06T 7/00; G16H 50/20

(52) U.S. Cl. CPC A61B 3/1216; A61B 3/0008; G06T 7/0012; G16H 50/20; G06T 2207/30041

(57) ABSTRACT

The present disclosure is directed to an imaging system and methods of use thereof. The system can include a light source configured to emit light to illuminate a retina of an eye with the light. The system can include one or more imaging devices configured to receive light from the retina via one or more polarizers to generate one or more spectropolarimetric images of the retina. The system can include a computing device configured to receive the one or more spectropolarimetric images of the retina, evaluate the one or more spectropolarimetric images, and identify one or more biomarkers indicative of a pathology.

### RELATED APPLICATIONS

This application is a bypass continuation patent application of PCT International Patent Application No. PCT/IB2023/000209, filed Apr. 20, 2023, which claims the benefit of and priority to U.S. Provisional Application Ser. No. 63/332,855, filed on Apr. 20, 2022, and U.S. Provisional Application Ser. No. 63/425,155, filed on Nov. 14, 2022, each of which are incorporated herein by reference in their entireties.

### FIELD

This disclosure relates to systems and methods for diagnosing disease by using optical techniques, in particular, systems and methods for diagnosing neurogenerative disease from retinal scan.

### BACKGROUND

Alzheimer's disease (AD) is a debilitating and fatal neurodegenerative disease. Confirmation of the disease is commonly performed post-mortem. Some existing conventional systems for diagnosis involve either highly invasive procedures or imaging devices that are often inaccessible or inappropriate due to cost, complexity, or the use of harmful radioactive tracers.

There is a need for a non-invasive detection system that is easily operable and accessible by clinicians for screening patient populations for early detection of AD-associated pathologies, diagnosis, and tracking of patient response to preventative or treatment interventions.

### SUMMARY

In some aspects, the techniques described herein relate to an imaging system including: a light source configured to emit light to illuminate a retina of an eye with the light; one or more imaging devices configured to receive light from the retina via one or more polarizers to be used for generating one or more spectropolarimetric images of the retina; and a computing device configured to receive the one or more spectropolarimetric images of the retina, evaluate the one or more spectropolarimetric images, and identify one or more biomarkers indicative of a pathology.

In some aspects, the techniques described herein relate to an imaging system, wherein the one or more imaging devices includes a snapshot camera.

In some aspects, the techniques described herein relate to an imaging system, wherein the one or more imaging devices includes a hyperspectral camera.

In some aspects, the techniques described herein relate to an imaging system, wherein the one or more polarizers are positioned between the one or more imaging devices and the eye, and wherein the one or more imaging devices are further configured to receive light from the retina via the one or more polarizers to generate the one or more spectropolarimetric images of the retina from polarized light passing through the one or more polarizers.

In some aspects, the techniques described herein relate to an imaging system, wherein the one or more polarizers are positioned between the light source and the eye, and wherein the light source is further configured to emit the light via the one or more polarizers to illuminate the retina of the eye with polarized light passing through the one or more polarizers.

In some aspects, the techniques described herein relate to a system, wherein the light source includes: one or more emitters configured to emit light towards one or more dichroic filters, wherein the one or more dichroic filters are configured to reflect light from the one or more emitters towards the retina of the eye, and wherein each of the one or more emitters are configured to emit the light through a corresponding dichroic filter of the one or more dichroic filters to illuminate the retina of the eye with the light.

In some aspects, the techniques described herein relate to a system, wherein the light source includes: a broadband emitter configured to emit light towards a tunable spectral sampling device, and wherein the tunable spectral sampling device is configured to filter the light from the broadband emitter towards the retina of the eye to illuminate the retina of the eye with the light.

In some aspects, the techniques described herein relate to a system, wherein the light source includes: a broadband emitter configured to emit light towards a filter, and wherein the filter is configured to filter the light from the broadband emitter towards the retina of the eye to illuminate the retina of the eye with the light.

In some aspects, the techniques described herein relate to a system, wherein the light source includes a broadband tunable emitter configured to emit light towards the retina of the eye to illuminate the retina of the eye with the light.

In some aspects, the techniques described herein relate to a system, wherein the light source includes: a broadband emitter configured to emit light towards a diffractive grating element, wherein the diffractive grating element is configured to reflect the light from the broadband emitter towards a filter, and wherein the filter is configured to reflect, based on one or more scanning elements, the light from the diffractive grating element towards of the eye to illuminate the retina of the eye with the light.

In some aspects, the techniques described herein relate to a system, wherein the light source includes: a scanning element configured to scan light emissions; and a broadband emitter configured to emit, based on the scanning element, light towards a prism, wherein the prism is configured to reflect the light from the broadband emitter towards a spectral filter, wherein the spectral filter is configured to filter the light from the prism towards an opening, and wherein the opening is configured to pass the light from the spectral filter towards the eye to illuminate the retina of the eye with the light.

In some aspects, the techniques described herein relate to a system, wherein the one or more imaging devices includes: one or more dichroic filters configured to reflect light from the retina of the eye towards one or more light monochromatic sensors, and wherein the one or more light monochromatic sensors are configured to sense the light from the one or more dichroic filters to generate the one or more spectropolarimetric images of the retina.

In some aspects, the techniques described herein relate to a system, wherein the one or more imaging devices includes: a tunable spectral sampling device configured to filter light from the retina of the eye towards a light monochromatic sensor, and wherein the light monochromatic sensor is configured to sense the light from the tunable spectral sampling device to generate the one or more spectropolarimetric images of the retina.

In some aspects, the techniques described herein relate to a system, wherein the one or more imaging devices includes: a filter configured to filter light from the retina of an eye towards a light monochromatic sensor, and wherein the light monochromatic sensor is configured to sense the light from the filter to generate the one or more spectropolarimetric images of the retina.

In some aspects, the techniques described herein relate to a system, wherein the one or more imaging devices includes: an optical element configured to filter light from the retina of the eye towards a dispersive optical element, wherein the dispersive optical element is configured to filter the light from the optical element towards a light monochromatic sensor, and wherein the light monochromatic sensor is configured to sense the light from the dispersive optical element to generate the one or more spectropolarimetric images of the retina.

In some aspects, the techniques described herein relate to a system, wherein the one or more imaging devices includes: a diffraction grating element configured to reflect light from the retina of the eye towards one or more scanning elements, wherein the one or more scanning elements reflect the light from the diffraction grating element towards a light monochromatic sensor, and wherein the light monochromatic sensor is configured to sense the light from the one or more scanning elements to generate the one or more spectropolarimetric images of the retina.

In some aspects, the techniques described herein relate to a system, wherein the one or more imaging devices includes: a prism configured to reflect light from the retina of the eye towards an opening, wherein the opening is configured to filter the light from the prism towards a light monochromatic sensor, and wherein the light monochromatic sensor is configured to sense the light from the opening to generate the one or more spectropolarimetric images of the retina.

In some aspects, the techniques described herein relate to a system, wherein the one or more imaging devices includes: an optical element configured to reflect light from the retina of the eye towards a filter, wherein the filter is configured to filter the light from the optical element towards a light monochromatic sensor, and wherein the light monochromatic sensor is configured to sense the light from the filter to generate the one or more spectropolarimetric images of the retina.

In some aspects, the techniques described herein relate to an imaging system including: a broadband light source configured to emit light at multiple wavelengths to illuminate an object; an imaging device including: a polarization filter array positioned to receive light reflected from the object and adjust a polarization of the light reflected from the object to generate polarimetric light; a spectral filter array positioned to receive polarized light and adjust a spectral state of the polarized light to generate spectropolarimetric light; a sensor positioned to receive the spectropolarimetric light, wherein the sensor is configured to simultaneously capture, from the spectropolarimetric light, one or more spatial components; one or more spectral components, and one or more polarimetric components associated with the object to generate one or more spectropolarimetric images; and a computing device configured to receive the one or more spectropolarimetric images of the object, evaluate the one or more spectropolarimetric images, and identify one or more biomarkers indicative of a pathology.

In some aspects, the techniques described herein relate to a system, wherein the spectral filter array further includes: a spectral sampling optical element positioned to receive or pass spectrally decomposed light from or to the polarization filter array or the sensor to generate the one or more spectropolarimetric images by optical focusing, collimation, refraction, diffraction or shaping.

In some aspects, the techniques described herein relate to a system, wherein the imaging device further includes a microlens array configured to receive light from the object and pass the light to the polarization filter array.

In some aspects, the techniques described herein relate to a system, wherein the spectral filter array includes spectral dispersing element positioned to receive the polarized light and disperse the polarized light by wavelength as it passes to the sensor.

In some aspects, the techniques described herein relate to a system, wherein the spectral dispersing element is attached to the polarization filter array.

In some aspects, the techniques described herein relate to a system, wherein the polarization filter array includes a plurality of polarization pixels that each correspond to a unique polarization angle.

In some aspects, the techniques described herein relate to a system, wherein the imaging device further includes a microlens array that houses the polarization filter array and the spectral filter array.

In some aspects, the techniques described herein relate to a method including: emitting, by an ocular imaging system, light to illuminate a retina of an eye with the light; receiving, by the ocular imaging system, light from the retina via one or more polarizers to generate one or more spectropolarimetric images of the retina; and evaluating, by the ocular imaging system, the one or more spectropolarimetric images, and identifying, by the ocular imaging system, one or more biomarkers indicative of a pathology.

In some aspects, the techniques described herein relate to a method, further including: receiving, by the ocular imaging system, light from the retina via the one or more polarizers to generate the one or more spectropolarimetric images of the retina from polarized light passing through the one or more polarizers.

In some aspects, the techniques described herein relate to a method, further including: emitting, by the ocular imaging system, the light via the one or more polarizers to illuminate the retina of the eye with polarized light passing through the one or more polarizers.

In some aspects, the techniques described herein relate to a method, wherein emitting the light includes: emitting, by the ocular imaging system, light from one or more emitters towards one or more dichroic filters; and reflecting, by the ocular imaging system, light from the one or more emitters to the one or more dichroic filters and towards the retina of the eye.

In some aspects, the techniques described herein relate to a method, wherein emitting the light includes: emitting, by the ocular imaging system, light towards a tunable spectral sampling device; and filtering, by the ocular imaging system, the light from a broadband emitter towards the retina of the eye to illuminate the retina of the eye with the light.

In some aspects, the techniques described herein relate to a method, wherein emitting the light includes: emitting, by the ocular imaging system, light towards a filter; and filtering, by the ocular imaging system, the light from the filter to a broadband emitter towards the retina of the eye to illuminate the retina of the eye with the light.

In some aspects, the techniques described herein relate to a method, wherein emitting the light includes: emitting, by the ocular imaging system, light towards the retina of the eye to illuminate the retina of the eye with the light.

In some aspects, the techniques described herein relate to a method, wherein emitting the light includes: emitting, by the ocular imaging system, light towards a diffractive grating element; reflecting, by the ocular imaging system, the light from a broadband emitter towards a filter; and reflecting, by the ocular imaging system, based on one or more scanning elements, the light from the filter to the diffractive grating element towards of the eye to illuminate the retina of the eye with the light.

In some aspects, the techniques described herein relate to a method, wherein emitting the light includes: emitting, by the ocular imaging system, based on a scanning element, light through a broadband emitter towards a prism; reflecting, by the ocular imaging system, the light from the broadband emitter through the prism and towards a spectral filter; filtering, by the ocular imaging system, the light from the prism through the spectral filter and towards an opening; and passing, by the ocular imaging system, the light from the spectral filter through the opening and towards the eye to illuminate the retina of the eye with the light.

In some aspects, the techniques described herein relate to a method, further including: reflecting, by the ocular imaging system, light from the retina of the eye through one or more dichroic filters towards one or more light monochromatic sensors; and causing, by the ocular imaging system, the one or more light monochromatic sensors to sense the light from the one or more dichroic filters to generate the one or more spectropolarimetric images of the retina.

In some aspects, the techniques described herein relate to a method, further including: filtering, by the ocular imaging system, light through a tunable spectral sampling device from the retina of the eye towards a light monochromatic sensor; and causing, by the ocular imaging system, the light from the tunable spectral sampling device to generate the one or more spectropolarimetric images of the retina.

In some aspects, the techniques described herein relate to a method, further including: filtering, by the ocular imaging system, light from the retina of an eye through a filter towards a light monochromatic sensor; and causing, by the ocular imaging system, the light monochromatic sensor to sense the light from the filter to generate the one or more spectropolarimetric images of the retina.

In some aspects, the techniques described herein relate to a method, further including: filtering, by the ocular imaging system, light from the retina of the eye through an optical element towards a dispersive optical element; filtering, by the ocular imaging system, the light from the optical element through the dispersive optical element towards a light monochromatic sensor; and causing, by the ocular imaging system, the light monochromatic sensor to sense the light from the dispersive optical element to generate the one or more spectropolarimetric images of the retina.

In some aspects, the techniques described herein relate to a method, further including: reflecting, by the ocular imaging system, light through a diffraction grating element from the retina of the eye towards one or more scanning elements; reflecting, by the ocular imaging system, the light from the one or more scanning elements to the diffraction grating element towards a light monochromatic sensor; and causing, by the ocular imaging system, the light monochromatic sensor to sense the light from the one or more scanning elements to generate the one or more spectropolarimetric images of the retina.

In some aspects, the techniques described herein relate to a method, further including: reflecting, by the ocular imaging system, light through a prism from the retina of the eye towards an opening; filtering by the ocular imaging system, the light through the opening from the prism towards a light monochromatic sensor; and causing, by the ocular imaging system, the light monochromatic sensor to sense the light from the opening to generate the one or more spectropolarimetric images of the retina.

In some aspects, the techniques described herein relate to a method, further including: reflecting, by the ocular imaging system, light through an optical element from the retina of the eye towards a filter; filtering by the ocular imaging system, the light through the filter from the optical element and towards a light monochromatic sensor; and causing, by the ocular imaging system, the light monochromatic sensor to sense the light from the filter to generate the one or more spectropolarimetric images of the retina.

In some aspects, the techniques described herein relate to a method including: emitting, by an ocular imaging system, light at multiple wavelengths to illuminate an object; receiving, by the ocular imaging system, light via a polarization filter array positioned to receive light through a reflected from the object and adjust a polarization of the light reflected from the object to generate polarimetric light; receiving, by the ocular imaging system, polarized light via a spectral filter array positioned to receive the polarized light and adjust a spectral state of the polarized light to generate spectropolarimetric light; sensing, by the ocular imaging system, the spectropolarimetric light via a sensor positioned to receive the spectropolarimetric light; extracting, by the ocular imaging system, from the spectropolarimetric light, one or more spatial components, one or more spectral components, and one or more polarimetric components associated with the object to generate one or more spectropolarimetric images; receiving, by the ocular imaging system, the one or more spectropolarimetric images of the object; evaluating, by the ocular imaging system, the one or more spectropolarimetric images; and identifying, by the ocular imaging system, one or more biomarkers indicative of a pathology.

### BRIEF DESCRIPTION OF DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 shows a block diagram of the ocular imaging system with the light source, the polarization control, and the imaging device.

FIG. 2A shows the polarimetric components.

FIG. 2B-2D show the polarimetric data.

FIG. 3A shows a ‘snapshot’ configuration in which the polarization is controlled using a filter array integrated with a hyperspectral imaging device and the ocular imaging system uses a broadband light source.

FIG. 3B provides examples of different hyperspectral sensors.

FIG. 4 shows a polarization and spectral filter arrays integrated with the sensor of the imaging device that is a ‘snapshot’ imaging device for simultaneously capturing images or data with spatial, spectral, and polarimetric components.

FIG. 5 shows a block diagram of the light source, the polarization control, and the imaging device contained within the ocular imaging system.

FIG. 6 shows a block diagram of the ocular imaging system with two polarizers.

FIG. 7 shows a block diagram of the light source, the polarization control, and the imaging device external to the ocular imaging system.

FIG. 8 shows a block diagram of the ocular imaging system with two external polarizers.

FIG. 9 shows an imaging device contained within the ocular imaging system and the light source and the polarization control independent of the ocular imaging system.

FIG. 10 shows a schematic of the ocular imaging system with two polarizers.

FIG. 11 shows a schematic of the ocular imaging system.

FIG. 12 shows the polarization controlled using a polarization filter wheel and the system makes use of the tunable light source and a monochromatic imaging device.

FIG. 13 shows examples of a tunable light source.

FIG. 14 shows the polarization controlled using fixed polarization filters and the ocular imaging system makes use of the broadband light source and the hyperspectral imaging device.

FIG. 15 and FIG. 16 shows the imaging device as a monochromatic camera and the light source as the tunable light source.

FIG. 17A shows a view of the eye.

FIG. 17B shows the regions of the optic disc to be segmented.

FIG. 18 shows a fundus camera.

FIG. 19 shows a flowchart of a method for processing spectropolarimetric images.

FIG. 20 depicts a block diagram of a computer-based system and platform.

FIG. 21 depicts a block diagram of a computer-based system and platform.

FIG. 22 illustrates a schematic of an implementation of a cloud computing/architecture.

FIG. 23 illustrates a schematic of an implementation of a cloud computing/architecture.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments. Embodiment examples are described as follows with reference to the figures. Identical, similar, or identically acting elements in the various figures are identified with identical reference numbers and a repeated description of these elements is omitted in part to avoid redundancies.

As the retina is an extension of the central nervous system, linked by the optic nerve directly to the brain, proteins produced in the brain as part of a neurological disease progression, such, for example, as beta amyloid and tau proteins indicative of Alzheimer's disease (AD), may be detected by ocular imaging. In individuals with Alzheimer's disease, both the amyloid and tau levels in the brain are elevated prior to the onset of symptoms. The levels of amyloid and tau are correlated in that subjects who develop AD tend to have biomarker evidence of elevated amyloid deposition biomarkers (which is detected via abnormal amyloid PET scan or low CSF Ab42 or Ab42/Ab40 ratio) as the first identifiable evidence of abnormality, followed by biomarker evidence of pathologic tau (which is detected via CSF phosphorylated tau, and Tau PET). This may be due to amyloid pathology inducing changes in soluble tau release, leading to tau aggregation later. In some embodiments, the systems and methods of the present disclosure can be used to detect, from a retinal scan, various disease biomarkers, such as, for example, Tau neurofibrillary tangles, Amyloid Beta deposits, soluble Amyloid Beta aggregates, or Amyloid precursor protein in the brain or the central nervous system to detect an existing neurological disease or an on-set of a neurological disease.

The detection of these biomarkers in the eye can be indicative of the presence or absence of these proteins in the brain or the central nervous system and corresponding risk of developing diseases. The eye can be examined using a variety of non-invasive light-based techniques to identify these health conditions because conditions affecting the optic nerve or retina can result in changes that induce different polarization changes in reflected light as a function of wavelength of the light. In some embodiments, the system and methods of the present disclosure generate spectropolarimetric images to reveal biomarkers of a neurological disease. The ability to detect information indicative of the presence or absence of biomarkers in brain tissue or the central nervous system using the polarimetric data from an ocular scan according to the present disclosure enables a quick and non-invasive diagnosis of a neurological disease such as Alzheimer's.

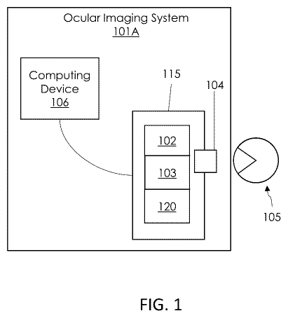

As shown in FIG. 1, disclosed is an ocular imaging system 101A for capturing one or more scans of the eye 105 for pathology detection or for diagnosing disease, such as Alzheimer's disease. The ocular imaging system 101A can generate one or more spectropolarimetric images of the eye 105, receive the one or more spectropolarimetric images of the eye 105, evaluate the one or more spectropolarimetric images, and identify one or more biomarkers indicative of a neurodegenerative pathology.

In some embodiments, the ocular imaging system 101A includes an imaging device 102, a light source 103 for illuminating the eye 105, an optical element 104 configured to direct the illumination light from the light source 103 to the eye 105, collect the light reflected, emitted, or returned from the eye, and a polarizer 120. In some embodiments, the ocular imaging system 101A includes a computing device 106 configured to receive the one or more spectropolarimetric images, evaluate the images, and identify one or more biomarkers indicative of a neurodegenerative disease. In some embodiments, one or more of the imaging devices 102, the light source 103, and the polarizer 120 can be in communication with a computing device 106 for obtaining and analyzing the spectropolarimetric images. The ocular imaging systems 101A-101L of the present disclosure may be presented as a stand-alone imaging system. In some embodiments, the ocular imaging systems 101A-101L of the present disclosure may be incorporated into a fundus camera or a similar ophthalmology examination device.

The light source 103 can be configured to illuminate the eye 105. The light source 103 may be a broadband light source 103, which emits a wide spectrum of light (e.g., UV, visible, near infrared, and/or infrared wavelength ranges), or it may be a narrowband light source 103 which emits a narrow spectrum or single wavelength of light. The light source 103 may emit a single continuous spectrum of light or it may emit a plurality of discontinuous spectra. In some embodiments, the wavelength composition of the light source and its intensity may be adjustable. In some embodiments, the light source 103 is configured to emit light only at wavelengths relevant for calculating the metrics indicative of systemic and localized diseases (e.g., Age Related Macular Degeneration, Retinopathy) and/or a metabolic state (e.g., oxygenation, blood circulation, bleaching of photoreceptors). In some embodiments, the light source 103 may comprises one or more super luminescent diodes (SLEDs), light emitting diodes (LEDs), xenon flashlight source, laser or light bulbs, a xenon lamp, a mercury lamp, or any other illuminator and light emitting elements. The light source 103 can include a single source of light or a combination of multiple sources of light of the same or different types described above.

In some embodiments, the light source 103 generates light having a known or predetermined polarization. The light source 103 may emit light circularly polarized, with one or more known polarization components (e.g., known spatial characteristics, frequencies, wavelengths, phases, and polarization states) or it may emit light with a random polarization (e.g., light that has a random mixture of waves having different spatial characteristics, frequencies, wavelengths, phases, and polarization states).

The polarizer 120 can comprise a polarization filter array comprising one or more polarization filters that transmit light waves of a specific polarization pass through while blocking light waves of other polarizations. In some embodiments, the polarizer 120 can be a mechanical, electromechanical, electrooptical device that rotates the transmitted polarization light using a mechanical, electromechanical, electrooptical driven mechanism (e.g., Pockels cells, rotating polarizers, liquid crystal device). In some embodiments, the polarizer 120 can provide linear, elliptical, or circular polarization. The polarizer 120 can reduce reflections, reduce atmospheric haze, and increase color saturation in images. The polarizer 120 can be an array of polarization filters (e.g., polarization filter array 127 as discussed herein) used to capture and measure different polarizations of incoming light on different pixels at the same time. The filter can provide polarization states at any one or more angles, such as, for example, 0, −45, 45, and 90 degrees. In some embodiments, the polarizers 120 can restrict the polarization of light that illuminates the eye 105 at any given time. In some embodiments, the polarizer 120 is an array of polarization filters each corresponding with one or more pixels of the imaging device 102. The polarizers 120 can be used to capture and measure different polarizations of incoming light sequentially by allowing light through the polarizer 120. In some embodiments, the polarizer may be combined with or otherwise work in combination with a spectral filter array comprising one or more spectral filters to limit the wavelengths of light received by the imaging devices 102 to the wavelengths relevant for calculating the metrics indicative of disease state. In some embodiments, the light source 103 includes the polarizers 120 to control or restrict the polarization of light that illuminates the eye 105 and the polarization of light reflected from the eye 105 that is received by the imaging device 102.

In some embodiments, the polarizer 120 can be placed between the light source 103 and the eye 105, between the eye 105 and the imaging device 102, or multiple polarizers 120 can be placed between both the light source 103 and the eye 105 and between the eye 105 and the imaging device 102. In some embodiments, the polarizer 120 can be integrated with the light source 103 or with the imaging devices 102 and in some embodiments, it can be separate. In some embodiments where the polarizer 120 is integrated with the imaging device 102. In some embodiments, the polarizer 120 may be placed both between the light source 103 and the eye 105, and between the eye 105 and the imaging device 102. In some embodiments, the polarizer 120 may be used to polarize the illumination source 103 or to polarize the collected light from the imaging device 102.

The imaging device 102 can be a device or sensor be configured to receive light returned from the eye 105. In some embodiments, the imaging device 102 can generate one or more spectropolarimetric images based on the light reflected from the eye. In some embodiments, the imaging device 102 may capture data that comprises spectral, spatial, and polarimetric components from which one or more polarimetric images can be constructed. In some embodiments, the imaging device 102 may capture data that comprises spectral, spatial, and polarimetric components of the same and different part of the object.

The imaging device 102 may be any optical assembly or sensor configured to collect and record light from the eye 105 or other parts of the fundus of the eye 105. The light source 103 may direct light toward the eye 105 and the imaging device 102 may be configured to collect and record light reflected, emitted, or returned from the eye 105. In some embodiments, the light source 103 can direct the light toward the eye 105 with the same optical assembly configured to collect light from the eye 105. In some embodiments, the light source 103 may direct light toward the eye 105 through a different optical path. The imaging device 102 can produce a measurement or the spectropolarimetric image of the eye 105 or any single component of the eye 105 illuminating the eye 105 with the light source 103 and collect the reflected, emitted or returned light from the eye 105 by the imaging device 102.

In some embodiments, the imaging device 102 can be a hyperspectral camera, snapshot hyperspectral camera, push broom hyperspectral camera, whiskbroom hyperspectral camera, staring hyperspectral camera, multispectral camera, spatial camera, or sensor configured to receive light returned from the eye 105 to generate or take one or more spectropolarimetric images of the eye 105, as will be discussed in more detail below. In some embodiments, the imaging device 102 can be a hyperspectral imaging sensor that can produce or generate the spectropolarimetric images. In some embodiments the light sensible sensor can be single pixels, a line of pixels or a matrix of pixels. In some embodiments, in addition to the imaging device 102, optical coherence tomography (OCT) or confocal scanning laser ophthalmoscopy (c-SLO) can be used to enhance and collect the spectropolarimetric images. In some embodiments, one or more single photon avalance diodes (SPADs), photomultiplier tubes (PMTs), or other photon sensing devices can also be used.

In some embodiments, the imaging device 102 includes a spectral sensor. In some embodiments, the spectral sensor can be a snapshot hyperspectral sensor, pushbroom hypespectral sensor, whiskbroom hyperspectral sensor, staring hypespectral sensor. In some embodiments, the spectral sensor can be a hyperspectral sensor, multispectral sensor, monochrome sensor, or an RGB sensor. In some embodiments, the spectral sensor may be a Fourier transform spectrometer used with a broadband light source. In some embodiments, any imaging system (e.g., systems 101A-101L) that allows for the collection of spectropolarimetric images may be used. In some embodiments, the spectral sensor may be a monochromatic sensor or other imaging device used with a tunable light source, and/or multiple light sources of different wavelengths, and/or a broadband light source with spectral filters to generate the spectral components. In some embodiments, the spectral sampling can be performed in the illumination optical path and/or in the detection optical path. In some embodiments, the spectral sampling can be performed using optomechanical (e.g., filter wheel), electro-optical (e.g., electro optical filter, liquid crystal), acusto-optical (e.g., acusto-optical filters) tunable filters device.

In some embodiments, the imaging device 102 can be any optical assembly that allows the record of an image of an object, a scene, or a sample. The imaging device 102 can be one or more microscopes (e.g., wide field, confocal), or optical coherence tomography system which contain imaging devices 102 (like a camera) configured to receive the spectropolarimetric images and communicate with a computer to transmit the spectropolarimetric images for analysis. The imaging device 102 can include one or more objective lenses and a camera sensor.

In some embodiments, a plurality of imaging devices 102 can be used to capture spectropolarimetric images at the same time or in sequence. In some embodiments, the plurality of imaging devices 102 capture the spectropolarimetric images with different, magnification, field of view, spatial resolution, and spectral resolution by using different imaging devices 102. In some embodiments, a first imaging device 102 could be coupled with the ocular imaging system 101A to produce a first spectropolarimetric image and then a second imaging device 102 could produce a second spectropolarimetric image. In some embodiments, the plurality of imaging devices 102 capture the spectropolarimetric images so that the spectropolarimetric image from a first imaging device 102 can be analyzed to identify spatial, spectral, or polarization components and determine which second imaging device 102 should be used and/or which locations or portions of the eye 105 to image with a second imaging device 102. In some cases, instead of using a second imaging device 102, the first imaging device 102 could be used with different settings (e.g., magnification or field of view) to capture a second spectropolarimetric image of the eye 105 with different spatial, spectral, or polarization components and resolution.

In some embodiments, the imaging device 102 comprises a scanning point spectrometer that generates the spectropolarimetric images in two dimensions. In some embodiments, the scanning spectrometer that can produce the spectropolarimetric images with both high spatial resolution and high spectral resolution with scanning optics and software. In some embodiments, the imaging device 102 comprises a line spectrometer that generates the spectropolarimetric images in one dimension (also referred to as a whisk broom imager). In some embodiments, the imaging device 102 comprises a matrix spectrometer that generates the spectropolarimetric images in two dimensions (also referred to as a push broom or staring imager). In some embodiments, a line spectrometer can be used to produce a one-dimensional spectropolarimetric image with a polarization measurement at each wavelength for each pixel along a line without scanning (e.g., 1×N), and a point spectrometer can produce a point ‘image’ (e.g., 1×1) without scanning. In some embodiments, a line spectrometer or point spectrometer can be used to produce higher dimensional images with spatial, spectral and or polarization scanning. In some embodiments, the imaging techniques allow the production of three-dimensional spectropolarimetric images in which a spectropolarimetric image is produced for each pixel in a three-dimensional volume.

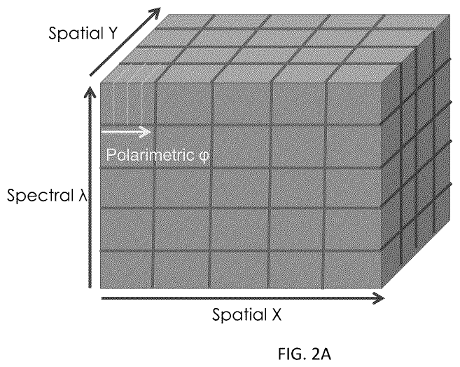

In some embodiments, a spectropolarimetric image comprises polarimetric components obtained from polarized light reflected, emitted, or returned from the eye. In some embodiments, one or more spectropolarimetric images can be generated by the imaging device 102 for analysis by the computing device 106. Referring now to FIG. 2A, in some embodiments, the spectropolarimetric image (also referred to as spectral-spatiopolarimetric images, spatial-spectropolarimetric images, or a spatial spectropolarimetry,) can include a spatial X component, a spatial Y component, a spectral λ component of wavelength, and a polarimetric φ component. The spectropolarimetric image can be a four-dimensional data or image (4-D image). In some embodiments, the spectropolarimetric image can be a 4-D image where the first and second dimensions are x-y dimensions, the third dimension is the spectral λ, and the fourth dimension is the polarization. In some embodiments, the 4-D data can be visualized as a 3-D cube noting the 3 dimensions of spatial and spectral (X, Y, λ), where each 3-D voxel of the cube is sliced to the different polarimetric components of the same spatial-spectral position. In some embodiments, the spectropolarimetric images include data elements of (X, Y, λ, φ).

In some embodiments, the spectropolarimetric image can be a two-dimensional spatial image with a polarization measurement of the light at two or more wavelengths for each image pixel (or a three-dimensional spatial image with a polarization measurement of the light at two or more wavelengths for each image voxel). In some embodiments, the spectropolarimetric image is a two-dimensional image having a polarimetric component and a spatial component with a single light intensity value for each image pixel, such as a two-dimensional image generated by a monochrome (grayscale) camera 132.

Now referring to FIG. 2B, in some embodiments, the spectropolarimetric image can identify each pixel on a x-y grid that encodes both spectrum (λ) and polarization (φ) parameters. In some embodiments, the spectropolarimetric image can comprise a 2-dimensional spatial array in which each pixel is associated with 2 or more polarimetric components measured at 2 or more different wavelengths.

Now referring to FIG. 2C, the polarimetric components may be represented in a 4×4 Mueller matrix that describes the reflectance of the eye 105 at various wavelengths. The input vector can be the incident light directed at the eye 105 from the light source 103 and the output vector can be the light reflected from the eye 105 to the imaging device 102. In some embodiments, the vectors are represented as a 4-element Stokes vector, or as other representations of the polarization of the incident and/or reflected light. Since the polarization state of the input and output light can be defined by four element vectors, the polarization components can be encoded on a 16-element Mueller Matrix with four polarization angles (for example, 0, −45, 45, 90) for both polarization state generator (PSG) (input light) and polarization state analyzer (PSA) (output light). Each element of the Mueller Matrix can indicate the reflectance of the eye 105 at various wavelengths at a specific polarization ratio of the input and output light. For example, the Mueller Matrix element M₀₀ corresponds to hyperspectral imaging without polarization. As shown in FIG. 2D, the Mueller matrix element M₁₃ indicates a reflectance spectrum λ at a particular ratio of polarization of input light and output light.

In some embodiments, the spectropolarimetric image can be a 3-dimensional spatial array generated by using a volumetric imaging technique such as optical coherence tomography (OCT). Each element in the spatial array may have arrays of wavelength and polarization values associated with it. In some embodiments, the spectropolarimetric image can include dimensionality based on plenoptic (light field) measurements or time-varying dynamic measurements.

The computing device 106 can receive and analyze spectropolarimetric images generated by the imaging device 102. In some embodiments, the computing device 106 can receive the one or more spectropolarimetric images from the imaging device 102. The imaging device 102 can be coupled to the computing device 106. In some embodiments, the outputs of the imaging devices 102 can be coupled to the computing device 106, such as a computer, PC, or laptop. The computing device 106 can receive the spectropolarimetric images from the imaging device 102. In some embodiments, the computing device 106 can be configured to control the settings of one or more of the imaging devices 102, including image settings as well as scanning and positioning settings.

In some embodiments, the computing device 106 can be configured to obtain, request, or receive a retinal image mosaic comprising the spectropolarimetric images of the eye 105. In some embodiments, the computing device 106 can analyze the one or more spectropolarimetric images to identify biomarkers indicative of a neurodegenerative pathology. In some embodiments, the computing device 106 can generate a digital representation indicative of a presence or absence of the biomarkers in the one or more regions of the eye 105.

The computing device 106 can receive or identify polarization components in the spectropolarimetric images. In some embodiments, the computing device 106 can identify the polarization of light in two or more orthogonal components and can be commonly represented in the form of a Mueller matrix. In some embodiments, the computing device 106 can identify polarization that is linear or circular. Common polarization measurements include depolarization, retardation (circular, linear, and elliptical), and diattenuation (circular and linear; also referred as dichroism). Other polarization measures included polarizance, anisotropy, and Q metric. In some embodiments, the computing device 106 can identify polarimetric components that can relate to an anatomical location. In some embodiments, the spectropolarimetric image can include polarimetric components related to certain pathologies, such as patterns, formations, or textures in the imaged region that can be seen based on the different wavelength or different polarizations at which the images are captured. In some embodiments, such pathologies may be observed or identified by the computing device 106.

The computing device 106 can use the polarimetric components to identify or characterize properties of tissue polarization and birefringence that are spectrally dependent. In some embodiments, the computing device 106 can generate or produce the spectropolarimetric images by combining the polarization component measurements for each wavelength at each pixel into a single intensity value for each wavelength at each pixel (or if the different polarization components are measured on different pixels, then by combining them into a single compound pixel). In some embodiments, the computing device 106 can generate or produce a purely spatial image from the spectropolarimetric images by combining the individual wavelength component measurements at each pixel into a single intensity value for that pixel.

The computing device 106 can perform wavelength calibration using a previously acquired spectrum of a mercury or mercury-argon lamp, or other light source 103 with well-defined spectral, spatial, polarimetric and photometric characteristics. The positions of wavelengths of the peaks in a mercury spectrum or any artificial certified reference sample have well-defined characterized wavelengths via NIST or other standards. The computing device 106 can compare the known wavelengths and the position of the peaks in the mercury or mercury-argon lamp or any reference sample spectrum with the spectrum measured by the spectral imaging device 102 and the pixels where those wavelengths and the position of those peaks appear in the measured spectrum.

The computing device 106 can use the comparison to allow for a pixel to wavelength mapping to be calculated for the spectropolarimetric image and the wavelengths of light in subsequent spectropolarimetric images to be known. The pixels in the spectropolarimetric images where the peaks of the mercury lamp are measured can be assigned to the known wavelengths of those peaks. By noting the pixels where each of the known mercury or mercury-argon lamp peaks is measured, the computing device 106 can calculate an interpolation function to map each spatial pixel to a wavelength value and this interpolation function can be used to correctly assign the wavelength values of each pixel in subsequent spectral measurements.

In some embodiments, the computing device 106 can tag or register different spectropolarimetric images to ensure alignment in space between the spectropolarimetric images. The computing device 106 can identify corresponding spatial components in two or more images and shifting (translating and/or rotating using either rigid or elastic transformations) the positions of the spectropolarimetric images so that those spatial components overlap in a co-registered coordinate system. The calculated shift for each spectropolarimetric image to the co-registered coordinate system can then be used to shift subsequent spectropolarimetric images.

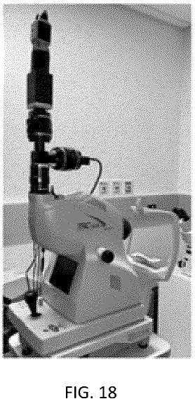

In some embodiments, the imaging device 102, the light source 103, and the polarizer 120 can be placed inside a housing 115 with an optical element 104 configured to direct light from the light source 103 to the eye 105, and light reflected, emitted, or returned from the eye 105 to the imaging device 102. In some embodiments, the housing 115 can be a fundus camera such as the one shown in FIG. 18. In some embodiments, the imaging device 102, light source 103, or polarizer 120 can be integrated into the housing 115. In some embodiments, the imaging device 102 can be in the form of a stand-alone device or a sensor configured to be attached to the housing 115. In some embodiments, the light source 103 and/or the polarizer 120 are attached to the ocular imaging system 101A. In some embodiments, the light source 103, the imaging device 102, and/or the polarizer 120 are separate from the housing 115. In some embodiments, the system 101A may further include an array of one or more spectral filters, either integrated with the polarizer 120 or as a standalone component of the system 101A.

In some embodiments, the ocular imaging system 101A includes a wavelength calibration source that emits narrowband light at one or more specific known wavelengths. The wavelength calibration source can be located within the housing 115 or placed externally to the housing 115. In some embodiments, the wavelength calibration source can be coupled to the light source 103. In some embodiments, the wavelength calibration source can be adjacent to the light source 103. The computing device 106 can receive a wavelength calibration signal from the imaging devices 102 that capture the light emitted by the wavelength calibration source. The computing device 106 can calculate a pixel to wavelength conversion for spectropolarimetric images from the corresponding wavelength calibration signal. Since the wavelength calibration source emits light at specific known wavelengths, the computing device 106 can assign the known wavelengths to the pixels on which the light falls. The computing device 106 can interpolate/extrapolate based on the known wavelengths to assign wavelength values to other pixels.

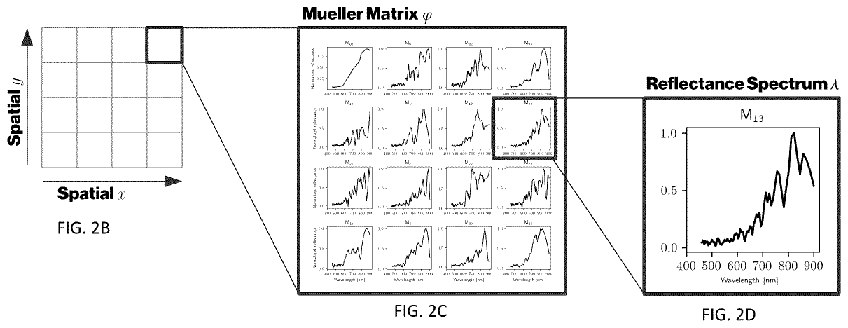

In some embodiments, as shown in FIG. 3A, the ocular imaging system 101B can include the light source 103, the polarizer 120, and the imaging device 102, which can be a snapshot spectra-spatial-polarimetric imaging device. A benefit of a snapshot spectra-spatial-polarimetric imaging device is that the spectral, polarimetric, and spatial components can be collected all at once.

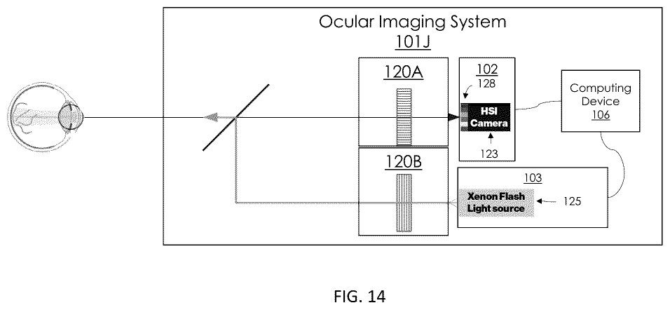

In FIG. 13 shows examples of embodiments to create spectrally tunable light sources 134A-134F that illuminates the sample, scene, or object (e.g., eye 105) for push broom, staring, multispectral, whiskbroom, hyperspectral imaging systems. In some embodiments, the spectral sampling can be obtained in the illumination optical path by the light source that includes any broadband continuous (e.g., Xenon lamp, Mercury lamp, LED) such as, for example, a xenon flash light source 125 including a tunable optical element or a dispersing optical element or a supercontinuum laser (e.g., tunable light sources 134B-134F) or discontinuous combinations of light sources (e.g. laser, LEDs, SLEDs) as for example, in tunable light source 134A.

In some embodiments, the tunable light source 134A can include a plurality of emitters 1302A-1302N (e.g., RGB, LED, laser, SLED, Optica fiber) that emit light. In some embodiments, the tunable light source 134A can include a plurality of dichroic filters 1304A-1304N through which light from the emitters 1302A-1302N can be emitted towards the eye 105. In some embodiments, the tunable light source 134A includes a dichroic filter for each emitter.

In some embodiments, the tunable light source 134B can include a broadband emitter 1306 (e.g., LED, laser, xenon lamp, mercury lamp, Xenon flashlight) that emit light. In some embodiments, the tunable light source 134B can include a tunable spectral sampling device 1308 (e.g., LCD, tunable filter, acousto-optical tunable filter, electro-optical tunable filter, opto-mechanical filter wheel) through which light from the broadband emitter can be emitted towards the eye 105.

In some embodiments, the tunable light source 134C can include a broadband emitter 1310 (e.g., LED, laser, xenon lamp, mercury lamp, Xenon flashlight) that emit light. In some embodiments, the tunable light source 134C can include a filter 1312 (e.g., scanning filter array, filter mosaic, diffractive optical element) through which light from the broadband emitter 1310 can be emitted towards the eye 105.

In some embodiments, the tunable light source 134D can include a broadband tunable emitter 1314 (e.g., supercontinuum tunable laser, swept source) that emit light towards the eye 105 (e.g., without any filters).

In some embodiments, the tunable light source 134E can include a broadband tunable emitter 1316 (e.g., supercontinuum laser, Ti-saphhire laser) that emit light. In some embodiments, the tunable light source 134E can include a diffractive grating filter 1318 that reflects light from the emitter 1316 towards the filter 1321. The filter 1321 can receive and reflect light from the diffractive grating filter 1318 towards the eye 105. In some embodiments, the tunable light source 134E is tuned based on a plurality of scanning elements 1320A-1320N that monitor the light being emitted by the emitter 1316.

The diffractive grating filter 1318 can diffract incident light beams and reflect different spectral wavelengths at different angles. For example, the grating filter 1318 can uncouple different wavelengths of the incident source by angular decomposition. Diffractive Optical Elements (DOE) can provide spectral or spatial sampling by phase delay between the different components of the light incident on it. This phase delay creates interference pattern that are constructive or destructive depending on the wavelength under examination.

In some embodiments, the tunable light source 134F can include a broadband tunable emitter 1322 (e.g., supercontinuum laser, Ti-saphhire laser) that emit light. In some embodiments, the tunable light source 134E can include a scanning element 1324 adjacent to the broadband tunable emitter 1322. In some embodiments, the tunable light source 134E includes a prism 1326 through which light from the broadband emitter 1322 travels towards a spectral filter 1327. In some embodiments, the tunable light source 134E can include an opening 1328 (e.g., pin hole) that passes light between the filter 1327 and the eye 105.

The prism 1326 can uncouple different wavelengths of the incident source by angular decomposition by using refraction. In some embodiments, gratings (e.g., filter 1318), prisms (e.g., prism 1326), and DOE can be used as a single element to separate wavelengths before a polarizer array that will encode polarization information.

In some embodiments, such as shown in FIG. 13, the spectropolarimetric image is obtained by active systems that spectrally sample and/or polarize the illumination source that illuminates the scene under observation with a non-constant, adjustable, tunable element, or light source and/or with an external signal is sent as a trigger to the light source or the imaging device (e.g., laser, LEDs, flash lamp that illuminates the sample only when requested by the system). Active system can be controlled by pure optical, opto-mechanical, electro-optical, or acusto-optical principles.

In some embodiments, the spectropolarimetric image of the scene is obtained by passive illumination systems that illuminates the scene independently to the state of the sample, imaging system, detection system (e.g., the sun that illuminates the earth).

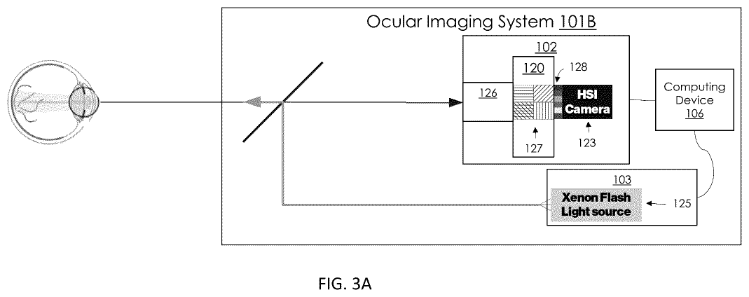

In some embodiments, as shown in FIG. 3A and FIG. 4, the polarizer 120 includes a polarization filter array 127 configured for mosaic-based polarization filtering across a plurality of polarization angles. The polarizer 120 can be positioned to receive light, focused or collimated, from a microlens array 126 and to pass the light to the spectral filter array 128 (e.g., spectral dispersing element, grating, diffractive optical element, or prisms array). In some embodiments, the polarizer 120 is internal to the imaging device 102 inserted in the illumination or detection optical paths. In some embodiments, the polarizer 120 is external to the imaging device 102 (e.g., attached between the microlens 126 and the spectral filter array 128).

In some embodiments, as shown in FIG. 3A and FIG. 4, the imaging device 102 can include the spectral filter array 128 and the polarizer 120, which can include the polarization filter array 127. The spectral filter array 128 can be positioned to receive light from the eye 105 and focus the light on the polarization filter array 127, which can polarize the light as it passes to the spectral filter array 128, which can disperse the light by wavelength as it passes to the imaging device 102. In some embodiments, the microlens 126 is internal to the imaging device 102 or to the sensor. In some embodiments, the spectral filter array 128 is external to the imaging device 102 (e.g., attached to the polarizer 120).

Since the imaging device 102 can measure light without distinguishing among colors, wavelength, or polarizations (e.g., ‘monochrome’ and ‘monopolar’ detectors), the polarization filter array 127 and the spectral filter array 128 advantageously separate the light by wavelengths and polarization. It should be noted that while the microlens and the spectral filter array are described as part of the polarizer 120, these elements may be provided as standalone elements configured to work in combination with the polarization array 127 of the polarizer 120.

In some embodiments, the microlens 126 can include 6×6 pixels formed by the 2×2 array of the polarization filter array 127 and the 3×3 array of the spectral filter array 128. The polarization filter array 127 can include a 2×2 array of 4 polarization pixels. Each polarization pixel can correspond to one of 4 polarization states/angles such as 0, 45, −45/135, and 90 degrees. Each polarization pixel can provide light of a different polarization to the 3×3 array of 9 spectral pixels of the spectral filter array 128. Since the 4 polarization pixels each correspond to 9 spectral pixels, the combination results in a 6×6 arrays of 36 pixels.

In some embodiments, the microlens 126 is a superpixel that receives light from a diffuser or the light source 103 that spreads or generates the light reflected from the eye 105. In some embodiments, the microlens 126 includes groups of pixels for each spatial element. In some embodiments, the microlens 126 can be positioned adjacent to the polarization filter array 127, such that the light from the eye 105 passes through the microlens 126.

The polarization filter array 127 can be configured for mosaic-based polarization filtering across a plurality of polarization angles. For example, the polarization filter array 127 can be configured for 4 polarization states or angles such as 0, 45, −45/135, and 90 degrees. Each polarization filter array 127 can polarize the same wavelength at a unique polarization. The polarization filter array 127 can be positioned adjacent to the spectral filter array 128, such that the light from microlens 126 passes through the polarization filter array 127 and onto the spectral filter array 128.

The spectral filter array 128 can be configured for mosaic-based snapshot multi or hyperspectral imaging to capture spectral measurements of polarization. Since the polarization filter array 127 filters the light in its polarization components, each spectral filter array 128 adjacent to the polarization filter array 127 can filter the same polarization at a specific wavelength. In some embodiments, an array of sensor pixels can be positioned underneath the spectral filter array 128 such that each pixel on the snapshot imaging device 102 will measure a different wavelength with a different polarization state of light.

The pixels within the spectral filter array 128 can each have a different spectral filter such that each pixel corresponds to different spectral measurements for each polarization. For example, the 9 pixels within each 3×3 array each can have a different spectral filter such that 9 different spectral measurements are made for each polarization. In some embodiments, 6×6 group of pixels of the microlens 126 can include 9 pixels of the spectral filter array 128 for measuring the same polarization at a different wavelength and 4 pixels of the polarization filter array 127 measuring the same wavelength corresponding to the spectral filter array 128 at a different polarization. In some embodiments, a 3×3 array of sensor pixels can be positioned underneath the spectral filter array 128 such that each pixel on the snapshot imaging device 102 will measure a different wavelength of light.

In some embodiments, to measure a spectropolarimetric image with 100 points in a 10×10 array, the polarizer 120 can include 3600 pixels in a 60×60 pixel configuration. Each group of 6×6 pixels can correspond to a point in the spectropolarimetric image, and each pixel in that 6×6 group would correspond with the spectral and polarization measurements as described above.

In some embodiments, the imaging device 102 includes an HSI camera 123 to capture spectropolarimetric images. In some embodiments, the HSI camera 123 includes sensors such as Charge Coupled Device (CCD) or a Complementary Metal-Oxide Semiconductor (CMOS) spectrometer to collect returned light and measure light intensity. In some embodiments, the imaging device 102 includes a spectral filter array 128 or a spectral dispersing element for filtering or dispersing the wavelengths of the reflected, emitted, or returned light. The spectral filter array 128 or spectral dispersing element can be positioned to receive light from the polarizer 120 and to pass the light to the HSI camera 123. In some embodiments, the spectral filter array 128 or spectral dispersing element is internal to the imaging device 102. In some embodiments, the spectral filter array 128 or spectral dispersing element is external to the imaging device 102 (e.g., attached before, between, or after the polarizer 120 and the HSI camera 123). In some embodiments, the imaging device 102 is a snapshot spectral imaging device (e.g., Ximea MQ022HG-IM-SM4X4-VIS or Cubert Firefly).

In some embodiments, as shown in FIG. 3A and FIG. 4, the imaging device 102 and/or the HSI camera 123 can include a microlens 126 and the polarizer 120, which can include the polarization filter array 127. The microlens 126 can be positioned to receive light from the eye 105 and focus the light on the polarization filter array 127, which can polarize the light as it passes to the spectral filter array 128, which can filter the light by wavelength as it passes to the imaging device 102 or to the sensor. In some embodiments, the microlens 126 is internal to the imaging device 102. In some embodiments, the microlens 126 is external to the imaging device 102 (e.g., attached to the polarizer 120).

In FIG. 3B are presented some examples of embodiments of the HSI camera 123A-123G to provide spectral sampling of the collected light from the imaging system, the scene, object, or sample under examination for push broom, staring, multispectral, whiskbroom, hyperspectral imaging systems.

In some embodiments, such as in HSI camera 123B, 123C, 123E, and/or 123F, the spectropolarimetric image is obtained by active systems that spectrally and/or polarize the collected light from the scene under observation with a non-constant, adjustable, tunable element, and/or with an external signal is sent as a trigger to the detection system or imaging device (e.g., liquid crystal tunable filters, filter wheels, acousto-optical filters, electro-optical filters, angle scanning diffractive gratings, prism). Active system can be controlled by pure-optical, opto-mechanical, electro-optical or acusto-optical principles.

In some embodiments, such as in HSI camera 123A, 123D, and 123G, the spectropolarimetric image from the scene is obtained by passive detection systems that collect, spectral sample, polarization sample and spatially sample the scene independently to the state of the sample, imaging system, detection system (e.g., a diffractive grating array, a prism, an array of filters, an array of prisms that disperse the light collected from a sample).

In some embodiments, such as shown in HSI camera 123A, 123D, and 123G, the spectral sampling can be obtained combining, optical standard elements such as lens, microlens arrays, cylindrical lens arrays, diffusers to focus, collimate, scatter and/or disperse the incoming light, with dispersing optical elements such as spectral filters arrays, spectral filter mosaic, diffractive optics.

In some embodiments, the HSI camera 123A can include a plurality of light monochromatic sensors 302A-302N (e.g., CCD, CMOS, photodiode) that sense light. In some embodiments, the HSI camera 123A can include a plurality of dichroic filters 304A-304N through which light from the imaging system 120 can be emitted towards the sensors 302A-302N. In some embodiments, the HSI camera 123A includes a dichroic filter for each sensor.

In some embodiments, the HSI camera 123B can include a light monochromatic sensor 306 (e.g., CCD, CMOS, photodiode) that senses light. In some embodiments, the HSI camera 123B can include a tunable spectral sampling device 308 (e.g., LCD, tunable filter, acousto-optical tunable filter, electro-optical tunable filter, opto-mechanical filter wheel) that can be tuned and through which light from the imaging system 120 travels to the sensor 306. In some embodiments, such as in HSI camera 123B, the spectral sampling can be obtained in the detection optical path by pure optical, opto-mechanical, electro-optical or acusto-optical devices that provide a continuous (e.g., dispersing elements as gratings, prisms, liquid crystal tunable filters) or discontinuous sampling of the incoming light (e.g., spectral filters wheels, spectral filter array).

In some embodiments, the spectral sampling can be done before the polarization sampling using optical tunable filters and a mosaic or an array of polarizers. In some embodiments, the polarization sampling can be done before the spectral sampling by pure-optical, electro-optical, acusto-optical or optomechanical polarizers such as Pockels cells, rotating polarizers, or liquid crystal tunable devices. The spectral sampling than can be carried out using diffractive optical elements, gratings, or prisms.

In some embodiments, the HSI camera 123C can include a light monochromatic sensor 310 (e.g., CCD, CMOS, photodiode) that senses light. In some embodiments, the HSI camera 123C can include a filter 312 (e.g., scanning filter array, filter mosaic, diffractive optical element) through which light from the imaging system 120 can travel towards the sensor 310.

In some embodiments, the HSI camera 123D can include a light monochromatic sensor 314 (e.g., CCD, CMOS, photodiode) that senses light. In some embodiments, the HSI camera 123D can include a dispersive optical element 316 (e.g., filter array, prism array, grating array, diffractive optical element) through which light can travel towards the sensor 314. In some embodiments, the HSI camera 123D can include an optical element 318 (e.g., optical focusing, shaping system, or collimating system) through which light can travel from the imaging system 120 to the dispersive optical element 316.

In some embodiments dispersing the light with the dispersing optical element 316 or an array such as a prism, a grating, or a tunable filter can be advantageous as there are fewer losses of collected light from tile joints between single elements of the filter array or mosaic and better optical quality.

In some embodiments, the optical element 316 can be inserted between the optical imaging device 102 to refocus, collimated and reshape the light before incidenting on the spectral sampling, polarization sampling or sensor pixels. The optical element 316 can be a microlens array, cylindrical lens array, spatial light modulator (SLM), liquid crystal devices, adaptive optics devices (e.g., mirror or lenses) and Micro Electro-Mechanical System (MEMS) coated with reflective coating can change phase, direction, and intensity of the incident light pattern. In some embodiments, the optical element 316 to refocus, collimates and/or reshape the incident light distribution can be used before, after and in between the polarization, spectral and spatial encoding and/or sampling of a spectropolarimetric camera, regardless of the order of the elements.

In some embodiments, the HSI camera 123E can include a light monochromatic sensor 320 (e.g., CCD, CMOS, PMT, photodiode) that senses light. In some embodiments, the HSI camera 123D can include one or more scanning elements 322A-322N through which light can travel towards the sensor 320. In some embodiments, the light travels through a first scanning element and then a second scanning element to reach the sensor 320. For example, as shown, the light can be reflected from one scanning element to another scanning element. In some embodiments, the scanning elements 322A-322N monitor the light being sensed by the sensor 320. In some embodiments, the sensor 320 senses the light based on the scanning elements 322A-322N that monitor the light from the eye 105. In some embodiments, the HSI camera 123E can include a diffraction grating element 324 (e.g., optical focusing, shaping system, or collimating system) through which light can travel from the imaging system 120 to the scanning elements 322A-322N.

The grating element 324 can diffract incident light beams and reflect or transmit different spectral wavelengths at different angles. For example, the grating element 324 can uncouple different wavelengths of the incident source by angular decomposition. Diffractive Optical Elements (DOE) can provide spectral or spatial sampling by phase delay between the different components of the light incident on it. This phase delay creates interference pattern that are constructive or destructive depending on the wavelength under examination.

In some embodiments, the HSI camera 123F can include a light monochromatic sensor 326 (e.g., CCD, CMOS, PMT, photodiode) that senses light. In some embodiments, the HSI camera 123F can include an opening 328 (e.g., filtering pinhole, spectral or scanning filtering pin hole) through which light is filtered and passed to the sensor 326. In some embodiments, the HSI camera 123F includes a prism 330 (e.g., scanning prism, fixed prism) through which light from the imaging system 120 travels to the opening 328.

The prism 330 can uncouple different wavelengths of the incident source by angular decomposition by using refraction. In some embodiments, gratings (e.g., grating element 324), prisms (e.g., prism 330), and DOE can be used as a single element to separate wavelengths before a polarizer array that will encode polarization information.

In some embodiments, the HSI camera 123G can include a light monochromatic sensor 332 (e.g., CCD, CMOS, PMT, photodiode) that senses light. In some embodiments, the HSI camera 123G can include a filter 334 (e.g., spectral filter array, filter array, filter mosaic) through which light travels to the sensor 332. In some embodiments, the HSI camera 123G can include an optical element 336 (e.g., microlens array, diffractive optics, focusing or shaping optical system, spatial light modulator) through which light from the imaging system 120 travels to the filter 334.

In some embodiments, for example as in HSI camera 123D or 123G, the small (e.g., grating element 324), prisms (e.g., prism 330), and DOE can be arranged as a mosaic or an array to encode the spectral sampling after the polarization sampling.

The imaging device 102 can capture the spectropolarimetric image with the spectral and polarimetric components for each spatial element in a single capture or exposure. The imaging device 102 can be configured to collect and process information from across the electromagnetic spectrum. The imaging device 102 can measure spatial distributions, spectral composition, and polarization of the light values based on how the pixels of the detector array are encoded among these parameters. Capturing the spectropolarimetric image in a single capture or exposure can be advantageous compared to capturing multiple exposures for different parameters (e.g., collecting all the data at a first wavelength on the first exposure, a second wavelength on a second exposure). Capturing the spectropolarimetric image in a single capture or exposure can be advantageous compared to capturing the data at a first polarization on the first exposure and at a second polarization on the second exposure. Collecting the data from a single capture or exposure can be faster, more accurate, more repeatable, more robust, time synchronized, reduce errors and artifacts, easier, and cheaper.

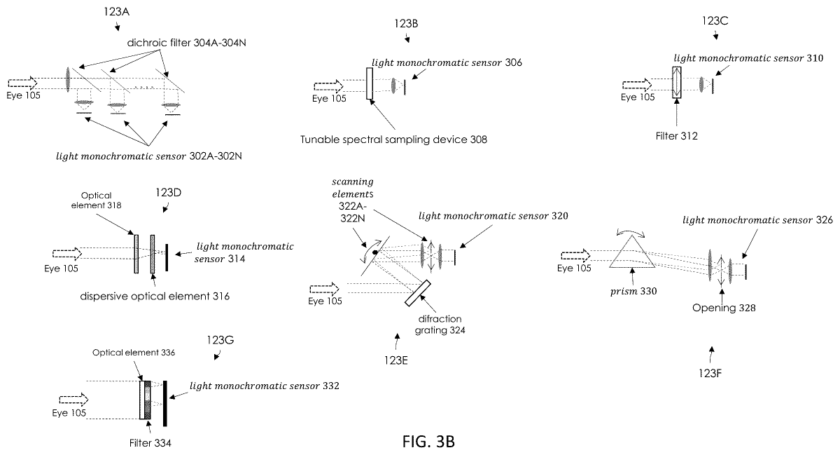

Referring now to FIG. 5, in some embodiments, the ocular imaging system 101C includes the polarizer 120 positioned at the output of the light source 103 (e.g., instead of the input of the imaging device 102) to control the polarization of the light incident upon the eye 105. Positioning the polarizer 120 between the imaging device 102 and the light source 103 enables the polarization 120 to control the polarization of light directed onto the eye 105 and thus reflected from the eye 105. The imaging device 102, light source 103, and polarizer 120 can be integrated into the housing 115 (e.g., a fundus camera) with the optical element 104 configured to direct light from the light source 103 to the eye 105, and light reflected, emitted, or returned from the eye 105 to the imaging device 102, and the polarizer 120 in the path of the light between the light source 103 and the imaging device 102. In some embodiments, the optical elements 104 are configured to couple with the imaging device 102 and polarizers 120 to add polarimetric measurement capability to an existing retinal viewing device without such capabilities.

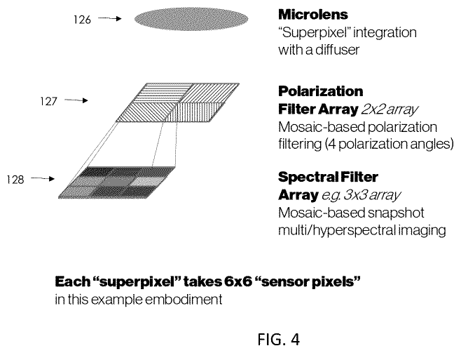

Referring now to FIG. 6, in some embodiments, the ocular imaging system 101D includes two polarizers 120, polarizer 120A and polarizer 120B coupled to the optical elements 104. In some embodiments, the optical elements 104 are configured to couple with the polarizer 120A and polarizer 120B to add polarimetric measurement capability to an existing retinal viewing device without such capabilities. This capability can be higher signal precision with the two-polarizer aligned, higher signal to noise ratio, polarimetric measures of both the incident and reflected, emitted and/or returned light, analysis of the dependencies of incoming and outgoing light by polarization of the incident light and reduce artifacts of stray light and reflected light from optical system shared components between the illumination and the detection. The polarizer 120A and polarizer 120B may have the same or different properties. Controlling the polarization of the output of the light source 103 and the input of the imaging device 102 simplifies calculating the polarimetric components compared to just controlling the polarization of one or the other. The polarizer 120A and polarizer 120B can measure different angles of polarization, where each angle can be measured in both aligned and crossed positions of the two polarizers. The polarizer 120A and polarizer 120B can be linear, circular, or elliptical. The computing device 106 can use the polarimetric components to identify or characterize properties of tissue polarization and birefringence that are spectrally dependent.

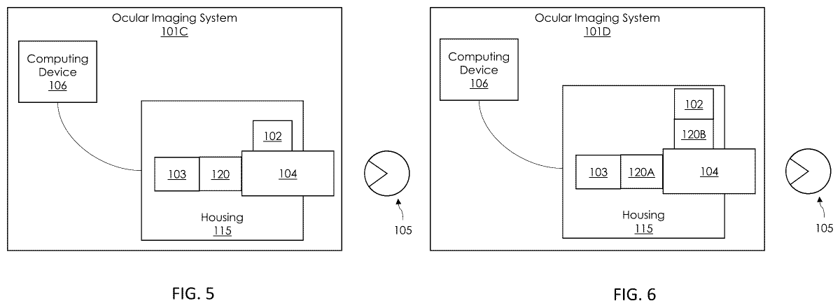

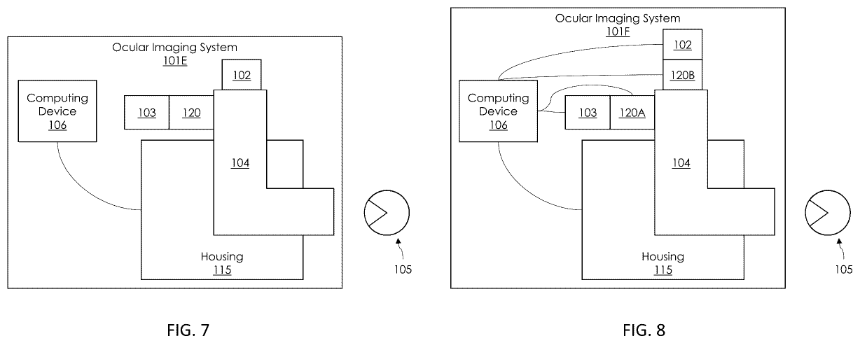

In some embodiments, as shown in FIG. 7, the ocular imaging system 101E includes the imaging device 102, light source 103, and polarizer 120 that are external to the housing 115. In some embodiments, the optical elements 104 are configured to couple with the imaging device 102 and polarizers 120 through external ports to add polarimetric measurement capability to an existing retinal viewing device without such capabilities. In some embodiments, as shown in FIG. 8, the ocular imaging system 101F includes the optical elements 104 configured to couple with two polarizers, polarizer 120A and polarizer 120B, positioned outside the housing 115.

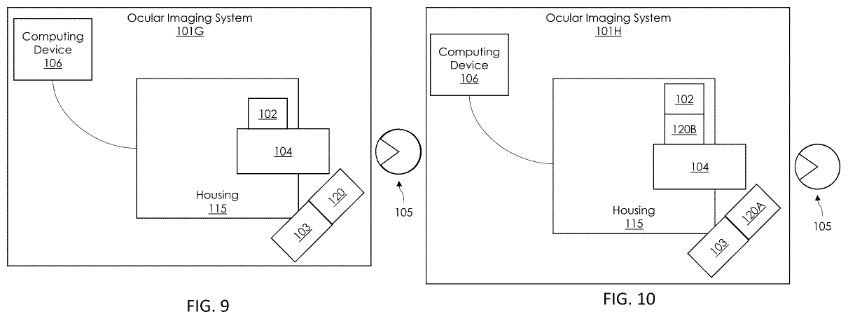

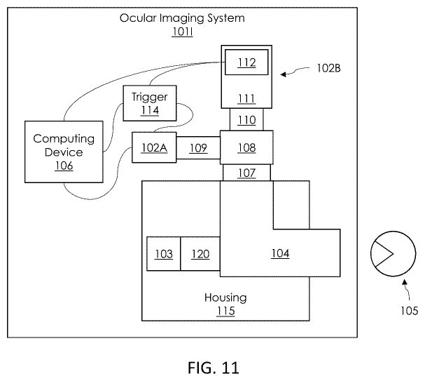

In some embodiments, as shown in FIG. 9, the ocular imaging system 101G includes the light source 103 and the polarizer 120 that are external to the housing 115 while the imaging device 102 is internal to the housing 115. In some embodiments, as shown in FIG. 10, the ocular imaging system 101H includes a polarizer 120A that is external to the housing 115 and the polarizer 120B that is internal to the housing 115. The two polarizers 120 may have the same or different properties. As shown in FIG. 9 and FIG. 10, the ‘hybrid’ configurations of having some of the components be inside the housing 115 while others are outside the housing 115 enables the components to be added to a variety of existing retinal viewing devices to add polarimetric measurement capabilities to existing retinal viewing devices without such capabilities.

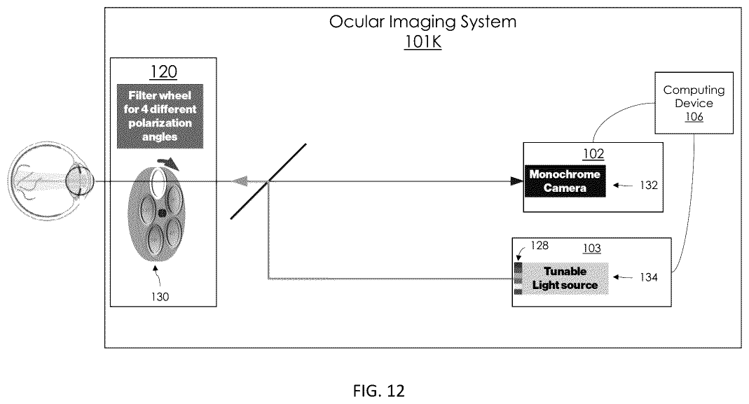

In some embodiments, as shown in FIG. 11, the spectropolarimetric images of the fundus of the eye 105 can be acquired with the ocular imaging system 101I, which can include imaging device 102A and imaging device 102B, a field lens 107, a beam splitter 108, a focusing lens 109, a macro lens 110, an optical spectrometer 111, a camera 112, and a trigger 114. The ocular imaging system 1011 includes control and triggering so that illumination, wavelength selection, polarization selection, and image capture can be automated and synchronized.

Illumination can be provided by the light source 103, which in some embodiments is a xenon flash lamp. The polarizer 120 can polarize the light emitted by the light source 103. In some embodiments, one or more extra polarizers may be placed at one or more points in the path of the light beam between the light source 103, the eye 105, the field lens 107, the beam splitter 108, lenses 109 and 110, and any components of the imaging devices 102A and 102B. The polarized light is directed toward the eye 105 via the optical element 104, which in some embodiments can include an objective lens. In some embodiments, the optical element 104 can reflect light from the eye 105 and direct it toward the field lens 107.

The field lens 107 can be positioned between the optical element 104 and the beam splitter 108. In some embodiments, the field lens 107 is coupled to an external camera port on the housing 115. In some embodiments, the field lens 107 is positioned outside the housing 115.

Light exiting the field lens 107 can be split by the beam splitter 108 into a first beam path directed through the focus lens 109 and a second beam path directed through the macro lens 110. In some embodiments, the beam splitter 108 is a partially reflective mirror, a beam redirecting device, a moveable mirror, or a beam redirector. In some embodiments, the beam splitter 108 is coupled to the field lens 107, the focus lens 109, and the macro or zoom lens 110. The beam redirecting device can redirect the light (in any ratio of time) collected by the optical element 104 to two or more imaging devices 102 in sequence. The beam splitter can divide the light between all the imaging device 102 at the same time (in either equal or unequal portions). In some embodiments, the beam splitter 108 is replaced by a beam redirecting device configured to send the light to each imaging device 102 in sequence (e.g., imaging device 102 not being directed to would get no light).

The first beam path of the beam splitter 108 can pass through the focusing lens 109 to the imaging device 102A configured to record spatial components. In some embodiments, the focusing lens 109 is coupled to the beam splitter 108. In some embodiments, the focusing lens 109 is a 60 mm focal length focusing lens. In some embodiments, the focusing lens 109 is coupled to the imaging device 102A. In some embodiments, the focusing lens 109 is positioned between the imaging device 102A and the beam splitter 108. In some embodiments, the focusing lens 109 focuses the light onto or into the imaging device 102A. The imaging device 102A and is coupled to the computing device 106 to provide the spectropolarimetric images to the computing device 106.

The second beam path of the beam splitter 108 can pass through the macro lens 110 to the imaging device 102B configured to record spectra components. In some embodiments, the macro lens 110 is a 60 mm focal length lens. In some embodiments, the macro lens 110 is coupled to the imaging device 102B. In some embodiments, the macro lens 110 is positioned between the imaging device 102B and the beam splitter 108. In some embodiments, the macro lens 110 focuses the light onto or into the imaging device 102B.

In some embodiments, the beam splitter 108 can split the light in any ratio of intensity collected by the optical element 104 between two or more imaging devices 102 coupled to the beam splitter 108 at the same time. In some embodiments, the ocular imaging system 101I can include two or more beam splitters 108 to be used in series to split and/or redirect the light between three or more imaging devices 102. The relative intensities of the beam paths from the beam splitter can be 50/50, 30/70, or other combinations to optimize the light intensity collected by each imaging device to generate the best images. In some embodiments, the ratio of light between the spatial camera and the spectral camera can be modulated depending on how much light is necessary for the spatial camera to generate images and for the spectral camera to generate images. For example, some types of images can ‘tolerate’ less light better than others, or some of the spectropolarimetric images are less important for the purpose of the analysis/diagnosis, which is why the ratio of splitting the light might can be adjusted accordingly.

In some embodiments, the imaging device 102B is a spectral imaging device that includes the spectrometer 111 configured to split the light from the beam splitter 108 into its spectral components. In some embodiments, the spectrometer 111 can be coupled to the beam splitter 108. In some embodiments, the spectrometer 111 is an imaging or non-imaging spectrometer. In some embodiments, the spectrometer 111 is a Specim VIR V10E or a Specim VNIR V10E.

In some embodiments, the imaging device 102B includes the camera 112 configured to measure the spectral components from the spectrometer 111. In some embodiments, the camera 112 is a PCO Pixelfly camera. The camera 112 can be configured to acquire or generate high resolution spectral measurements of the light split by the spectrometer 111. The spectral measurements include the wavelength components of light of a point or line across the posterior surface of the fundus. The spectral measurements may be of a narrow or broad range of wavelengths and may be of a continuous or discontinuous set of wavelengths or wavelength ranges (e.g., spectral bands). The spectral measurement can be made based on emitted radiation, fluorescence, absorbance, transmission, reflectance, wavelength shifts (such as Raman spectroscopy which measure a wavelength shift relative to a narrow-band light source 103 such as a laser), or other spectral, spatial, optical, or polarimetric properties. The computing device 106 can receive or identify the spectral measurements produced by the camera 112.

In some embodiments, the ocular imaging system 1011 includes the trigger 114 to synchronize the imaging devices 102 or light source 103. The trigger 114 may be coupled to the imaging devices 102 or light source 103. The trigger 114 can control the light source 103 to synchronize the imaging devices 102. The trigger 114 may be coupled to the computing device 106, which can control the trigger 114. In some embodiments, the computing device 106 can transmit a triggering signal to the trigger 114 to cause the imaging devices 102 to capture time synchronized images. For example, the trigger 114 can cause the light source 103 (e.g., xenon flash lamp, supercontinuum laser, mercury) to emit light (e.g., flash, radiation emission), which can trigger the acquisition of spectropolarimetric images by the imaging devices 102.

In some embodiments, the trigger 114 can control the polarizer 120. In some embodiments, the computing device 106 can transmit a triggering signal to the trigger 114 to cause the polarizer 120 to filter or polarize light. For example, the trigger 114 can cause the polarizer 120 to move into position or generate an electric field to polarize light passing through the polarizer 120, also to apply a desired filter.

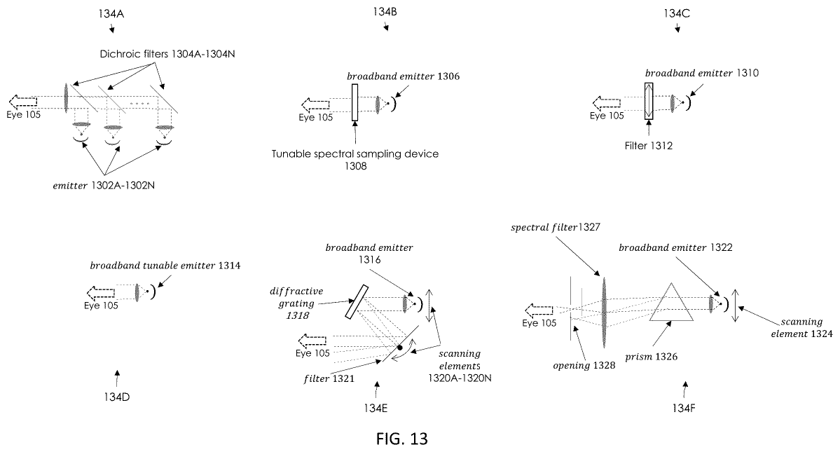

As shown in FIG. 12, the ocular imaging system 101K can include a filter wheel 130 configured to select light filtering, a monochrome camera 132 configured to capture an amount of light, and a tunable light source 134 configured to emit light at a selected wavelength on the eye 105, such as for example, by using the spectral filter array 128. The ocular imaging system 101J can enable addition of polarimetric measurement capability to an existing retinal viewing device without such capabilities. In some embodiments, the imaging device 102 includes the monochrome camera 132 (also referred to as monochromatic camera or grayscale camera), which is configured to detect or capture an amount of light.

In some embodiments, the light source 103 includes the tunable light source 134, which is configured to modify or select the wavelength of emitted light. The tunable light source 134 can emit light through the spectral filter array 128, which can be configured to filter light by wavelength to optimize the light for capture by the monochrome camera 132. In some embodiments, the tunable light source 134 is internal to the light source 103. In some embodiments, the tunable light source 134 is external to the light source 103 (e.g., attached to the light source 103).

In some embodiments, the polarizer 120 includes the filter wheel 130, which can include one or more polarization filters configured to control the polarization of light illuminating and reflecting from the eye 105. In some embodiments, the filter wheel 130 can include one or more polarization filters to filter the output of the light source 103 or to filter the light reflected from the eye 105, or to filter light entering one or more imaging devices 102, so that only polarized light is received by the one or more imaging devices 102. In some embodiments, in addition to or instead of the one or more polarization filters, the filter wheel 130 can include one or more spectral filters configured to filter light by wavelength. The filter wheel 130 may be optically, mechanically, electrically, or acoustically adjustable or switchable (e.g., a filter wheel, acousto-optic tunable filter, or liquid crystal variable retarder (LCVR)) such that one or more wavelengths or wavelength ranges of light can be selected to pass through the filter at the same time or in sequence.

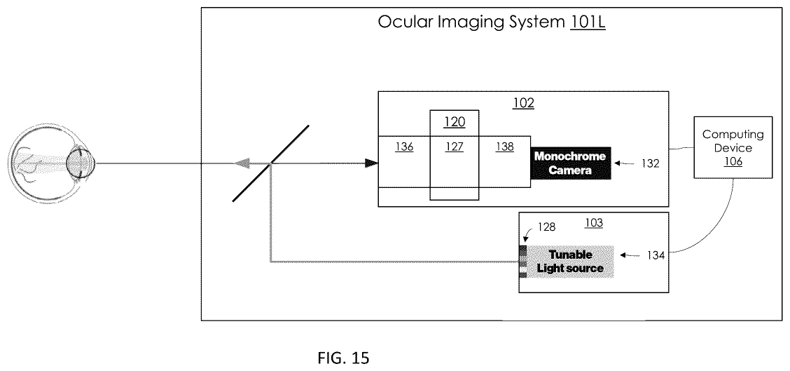

As shown in FIG. 14, the ocular imaging system 101J can include a pair of polarizers, polarizer 120A and polarizer 120B that are orthogonal to each other such that the polarizer 120B controls the polarization of the light exiting the light source 103 (e.g., a Xenon flash) and the polarizer 120A controls the polarization of the light received by the HSI camera 123 through the spectral filter array 128. In some embodiments, the spectral filter array 128 is internal to the imaging device 102. In some embodiments, the spectral filter array 128 is external to the imaging device 102 (e.g., attached between the polarizer 120A and the HSI camera 123).

In some embodiments, polarizer 120B can include the filter wheel 130 configured to adjust polarization of light illuminating the eye 105 from the light source 103. In some embodiments, polarizer 120A can include the filter wheel 130 configured to receive the light reflected from the eye 105 toward the imaging device 102. Controlling the polarization of the output of the light source 103 and the input of the imaging device 102 simplifies calculating the polarimetric components compared to just controlling the polarization of one or the other. The polarizer 120A and polarizer 120B can measure different angles of polarization, where each angle can be measured in both aligned and crossed positions of the two polarizers. In some embodiments, however, the polarizer 120A and polarizer 120B can be set for the same angle of polarization to increase polarimetric performances. The polarizer 120A and polarizer 120B can be linear, circular, or elliptical.

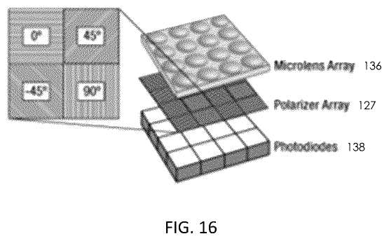

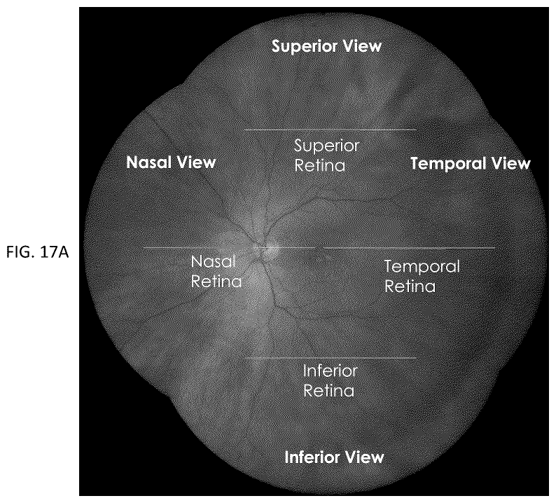

In some embodiments, as shown in FIG. 15 and FIG. 16, the imaging device 102 of the ocular imaging system 101L can include the microlens array 136 configured to focus light from the eye 105 onto the polarization filter array 127 of the polarizer 120. The imaging device 102 of the ocular imaging system 101L can include the photodiodes 138 configured to detect light from the polarizer array 127. The ocular imaging system 101L can offload the spectral selection to the tunable light source 134 that delivers the spectral components (e.g., light at a selected wavelength) one at time instead delivering them all at once.

The microlens array 136 can focus the light onto the polarization filter array 127, and onto the sensor pixels of the imaging device 102. In some embodiments, the microlens array 136 can be a plurality of the microlens 126. In some embodiments, the microlens array 136 is internal to the imaging device 102. In some embodiments, the microlens array 136 is external to the imaging device 102 (e.g., attached to the polarizer 120).

Each filter of the polarization filter array 127 can filter a respective polarization. In some embodiments, the monochrome camera 132 includes 2×2 groups of pixels for each spatial element and the polarization filter array 127 causes each pixel of the 2×2 polarization filter array 127 to provide a different polarization. In particular, the 2×2 array shows how 4 filters can be used to provide 4 different polarizations. The polarization filter array 127 can be repeated 4 times to make up the 4×4 polarization array above the photodiodes 138. In some embodiments, the polarizer 120 comprising the polarization filter array 127 is internal to the imaging device 102. In some embodiments, the polarizer 120 comprising the polarization filter array 127 is external to the imaging device 102 (e.g., attached between the microlens array 136 and the photodiodes 138).

In some embodiments, the photodiodes 138 are the sensor pixels of the monochrome camera 132. The monochrome camera 132 can be configured with sensor pixels allocated to detect spectral, polarimetric, or spatial parameters. In some embodiments, the photodiodes 138 are internal to the monochrome camera 132. In some embodiments, the photodiodes 138 are external to the monochrome camera 132 (e.g., attached between the polarizer 120 and the monochrome camera 132).

In some embodiments, the monochrome camera 132 can generate a spectropolarimetric image that is a two-dimensional image with a single light intensity value for each sensor pixel. In some embodiments, the spectropolarimetric image is generated by using the tunable light source 134 (e.g., sequential electrical switching of the tunable light source 134 spectrum composed of multiple LEDs, or with a supercontinuum laser with an acousto-optical tunable filter) and the monochrome camera 132 captures polarization images at multiple illumination wavelengths.

In some embodiments, the computing device 106 can produce or generate the spectropolarimetric images by using the monochrome camera 132 (or scanning a single point sensor) and sequentially measuring different wavelengths by illuminating the eye 105 with light of different wavelengths (by changing the light source 103 and/or the wavelengths of light emitted from the light source 103, and/or by changing the spectral filter array 128 used at the output of the light source 103) or by placing the spectral filter array 128 at any point in the optical path between the light source 103 and the eye 105 to filter the light received by the imaging device 102, while controlling the polarization of the light in one or more of the spectropolarimetric images.

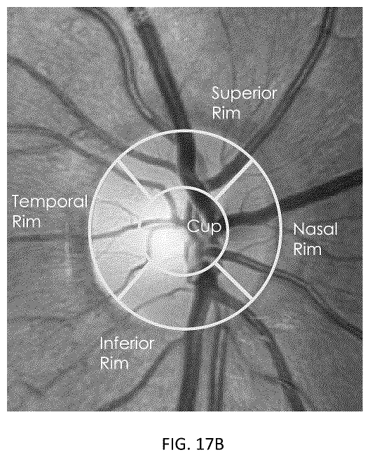

Using the ocular imaging systems described herein, various views of fundus can be acquired as shown in FIG. 17A. In some embodiments, the ocular imaging systems can be used to image the fundus of the eye 105 by providing broadband illumination and imaging optics, including an integrated or external camera to capture the spectropolarimetric image of the fundus of the eye 105. In some embodiments, the ocular imaging systems can provide illumination and image the posterior of the eye 105 (using an internal integrated camera).

As shown in FIG. 17A, the spectropolarimetric images can be regionally segmented to identify pixels in the various components of the eye 105, including the optic disc (nerve head), retina, and fovea. The computing device 106 can identify or determine the existence of one or more AD-associated pathologies, including but not limited to protein aggregates, the protein aggregates including at least one of: Tau neurofibrillary tangles, Amyloid Beta deposits, soluble Amyloid Beta aggregates, or Amyloid precursor protein. In some embodiments, the computing device 106 can use the first imaging modality to identify the locations of blood vessels in the eye 105 (e.g., based on spatial components in a spectropolarimetric image and/or by detecting blood flow from the spectropolarimetric image). In some embodiments, the computing device 106 can use the second imaging modality to analyze the spectral components of the blood vessels where the neurological disorders or pathologies may be more likely to be evident.

In some embodiments, the computing device 106 can segment the regions within the optic disc to identify more specific components, including a temporal rim, nasal rim, inferior rim, superior rim, and cup regions as shown in FIG. 17B. The segmentation of the imaged components of the eye 105 can be performed in a variety of ways. In some embodiments, the segmentation can be performed manually. In some embodiments, the computing device 106 can perform the segmentation with an automated segmentation algorithm.

In some embodiments, the housing 115 described herein is a fundus camera as shown in FIG. 18. In some embodiments, the fundus camera is a Topcon NW8, EX, or DX. In some embodiments, the fundus camera includes an external camera port.

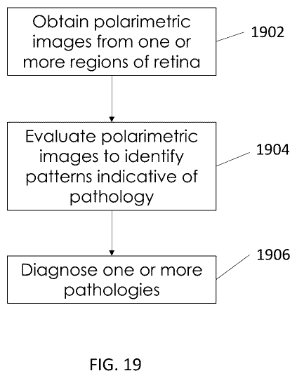

FIG. 19 illustrates a method for processing spectropolarimetric images that include polarimetric components. At step 1902, the imaging devices 102 capture the spectropolarimetric images from one or more regions of the eye 105. The computing device 106 can receive or maintain the spectropolarimetric images generated by the imaging device 102.

At step 1904, the computing device 106 can use the polarimetric components associated with the images to determine or identify one or more patterns indicative of pathology. In some embodiments, the computing device 106 can detect protein aggregates of Aβ, tau, phosphorylated tau and other neuronal proteins indicative of a neurodegenerative disease, in particular Alzheimer's disease. In some embodiments, the detected protein aggregates can include at least one of Tau neurofibrillary tangles, Amyloid Beta deposits or plagues, soluble Amyloid Beta aggregates, or Amyloid precursor protein. These detected proteins can be indicative of a pathology in the brain as they can be correlated to brain amyloid and/or brain tau.

At step 1906, the computing device 106 diagnoses one or more pathologies. The computing device 106 allows for the identification of at-risk populations, diagnosis, and tracking of patient response to treatments. In some embodiments, the computing device 106 can detect the existence of one or more of AD associated pathologies or pathologies associated with neurodegenerative diseases (e.g., Parkinson's disease (PD), amyotrophic lateral sclerosis (ALS), multiple sclerosis, Prion disease, motor neuron diseases (MND), Huntington's disease (HD), Spinocerebellar ataxia (SCA), Spinal muscular atrophy (SMA), cerebral amyloid angiopathy (CAA), other forms of dementia, and similar diseases of the brain or the nervous system). In some embodiments, the computing device 106 can detect other conditions in and related to the eye 105 such as age-related macular degeneration and glaucoma.

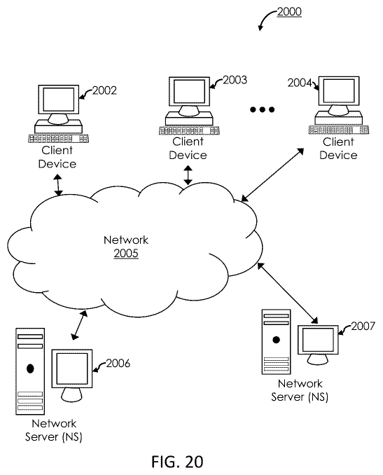

FIG. 20 depicts a block diagram of a computer-based system and platform 2000 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 2000 may be configured to manage a large number of members and concurrent transactions, as detailed herein. In some embodiments, the exemplary computer-based system and platform 2000 may be based on a scalable computer and network architecture that incorporates various strategies for assessing the data, caching, searching, and/or database connection pooling. An example of the scalable architecture is an architecture that is capable of operating multiple servers.

In some embodiments, referring to FIG. 20, member computing device 2002, member computing device 2003 through member computing device 2004 (e.g., clients) of the exemplary computer-based system and platform 2000 may include virtually any computing device capable of receiving and sending a message over a network (e.g., cloud network), such as network 2005, to and from another computing device, such as servers 2006 and 2007, each other, and the like. In some embodiments, the member devices 2002-2004 may be personal computers, multiprocessor systems, microprocessor-based or programmable consumer electronics, network PCs, and the like. In some embodiments, one or more member devices within member devices 2002-2004 may include computing devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile computing device, and the like. In some embodiments, one or more member devices within member devices 2002-2004 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, Wi-Fi, WiMAX, CDMA, satellite, Bluetooth, ZigBee). In some embodiments, one or more member devices within member devices 2002-2004 may include may run one or more applications, such as Internet browsers, mobile applications, voice calls, video games, videoconferencing, and email, among others. In some embodiments, one or more member devices within member devices 2002-2004 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 2002-2004 may be specifically programmed by either Java, .Net, QT, C, C++ and/or other suitable programming language. In some embodiments, one or more member devices within member devices 2002-2004 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 2005 may provide network access, data transport and/or other services to any computing device coupled to it. In some embodiments, the exemplary network 2005 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 2005 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 2005 may include and implement, as an alternative or in conjunction with one or more of the above, a WiMAX architecture defined by the WiMAX forum. In some embodiments and, optionally, in combination of any embodiment described above or below, the exemplary network 2005 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 2005 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 2005 may also include mass storage, such as network attached storage (NAS), a storage area network (SAN), a content delivery network (CDN) or other forms of computer or machine-readable media.

In some embodiments, the exemplary server 2006 or the exemplary server 2007 may be a web server (or a series of servers) running a network operating system, examples of which may include but are not limited to Microsoft Windows Server, Novell NetWare, or Linux. In some embodiments, the exemplary server 2006 or the exemplary server 2007 may be used for and/or provide cloud and/or network computing. Although not shown in FIG. 20, in some embodiments, the exemplary server 2006 or the exemplary server 2007 may have connections to external systems like email, SMS messaging, text messaging, ad content providers. Any of the features of the exemplary server 2006 may be also implemented in the exemplary server 2007 and vice versa.

In some embodiments, one or more of the exemplary servers 2006 and 2007 may be specifically programmed to perform, in non-limiting example, as authentication servers, search servers, email servers, social networking services servers, SMS servers, IM servers, MMS servers, exchange servers, photo-sharing services servers, advertisement providing servers, financial/banking-related services servers, travel services servers, or any similarly suitable service-base servers for users of the member computing devices 801-2004.

In some embodiments and, optionally, in combination of any embodiment described above or below, for example, one or more exemplary computing member devices 2002-2004, the exemplary server 2006, and/or the exemplary server 2007 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

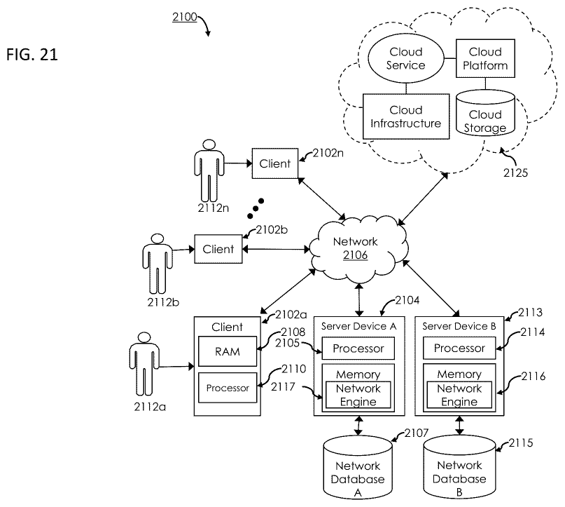

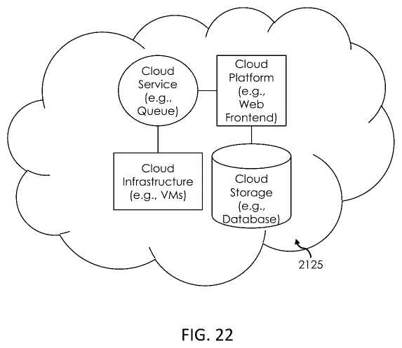

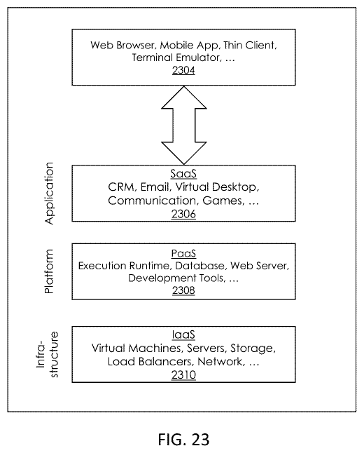

FIG. 21 depicts a block diagram of another exemplary computer-based system and platform 2100 in accordance with one or more embodiments of the present disclosure. However, not all these components may be required to practice one or more embodiments, and variations in the arrangement and type of the components may be made without departing from the spirit or scope of various embodiments of the present disclosure. In some embodiments, the member computing device 2102a, member computing device 2102b through member computing device 2102n shown each at least includes a computer-readable medium, such as a random-access memory (RAM) 2108 coupled to a processor 2110 or FLASH memory. In some embodiments, the processor 2110 may execute computer-executable program instructions stored in memory 2108. In some embodiments, the processor 2110 may include a microprocessor, an ASIC, and/or a state machine. In some embodiments, the processor 2110 may include, or may be in communication with, media, for example computer-readable media, which stores instructions that, when executed by the processor 2110, may cause the processor 2110 to perform one or more steps described herein. In some embodiments, examples of computer-readable media may include, but are not limited to, an electronic, optical, magnetic, or other storage or transmission device capable of providing a processor, such as the processor 2110 of client 2102a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript.

In some embodiments, member computing devices 2102a through 2102n may also comprise a number of external or internal devices such as a mouse, a CD-ROM, DVD, a physical or virtual keyboard, a display, or other input or output devices. In some embodiments, examples of member computing devices 2102a through 2102n (e.g., clients) may be any type of processor-based platforms that are connected to a network 2106 such as, without limitation, personal computers, digital assistants, personal digital assistants, smart phones, pagers, digital tablets, laptop computers, Internet appliances, and other processor-based devices. In some embodiments, member computing devices 2102a through 2102n may be specifically programmed with one or more application programs in accordance with one or more principles/methodologies detailed herein. In some embodiments, member computing devices 2102a through 2102n may operate on any operating system capable of supporting a browser or browser-enabled application, such as Microsoft™, Windows™, and/or Linux. In some embodiments, member computing devices 2102a through 2102n shown may include, for example, personal computers executing a browser application program such as Microsoft Corporation's Internet Explorer™, Apple Computer, Inc.'s Safari™, Mozilla Firefox, and/or Opera. In some embodiments, through the member computing client devices 2102a through 2102n, user 2112a, user 2112b through user 2112n, may communicate over the exemplary network 2106 with each other and/or with other systems and/or devices coupled to the network 2106. As shown in FIG. 21, exemplary server devices 2104 and 2113 may include processor 905 and processor 2114, respectively, as well as memory 2117 and memory 2116, respectively. In some embodiments, the server devices 2104 and 2113 may be also coupled to the network 2106. In some embodiments, one or more member computing devices 2102a through 2102n may be mobile clients.

In some embodiments, at least one database of exemplary databases 2107 and 2115 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

In some embodiments, the exemplary inventive computer-based systems/platforms, the exemplary inventive computer-based devices, and/or the exemplary inventive computer-based components of the present disclosure may be specifically configured to operate in a cloud computing/architecture 2125 such as, but not limiting to: infrastructure a service (IaaS) 2310, platform as a service (PaaS) 2308, and/or software as a service (Saas) 2306 using a web browser, mobile app, thin client, terminal emulator or other endpoint 2304. FIGS. 7 and 8 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

In some embodiments, the pathology information of a patient can be compared to personal history of the same patient to see a progression (regression). The progression (regression) of the patient can also be compared to other population cohorts and their historical progression (regression).

In some embodiments, spectropolarimetric information can be uncoupled in its fundamental elements by a linear and not linear combination of spectra using spectral unmixing algorithms, regressions, and/or prediction.

The server can implement the machine learning algorithm by way of one or more neural networks. The machine learning algorithm can include logistic regression, variational autoencoding, convolutional neural networks, transformers, or other statistical techniques used to identify and discern neurodegenerative disease-associated pathologies. The machine learning algorithm can also use spectral scattering models, spectral unmixing models, other scattering models, or optical physics models that are validated a priori. The neural network may comprise a plurality of layers, some of which are defined and some of which are undefined (or hidden). The neural network is a supervised learning neural network.

In some embodiments, the neural network may include a neural network input layer, one or more neural network middle hidden layers, and a neural network output layer. Each of the neural network layers include a plurality of nodes (or neurons). The nodes of the neural network layers are connected, typically in series. The output of each node in a given neural network layer is connected to the input of one or more nodes in a subsequent neural network layer. Each node is a logical programming unit that performs an activation function (also known as a transfer function) for transforming or manipulating data based on its inputs, a weight (if any) and bias factor(s) (if any) to generate an output. The activation function of each node results in a particular output in response to particular input(s), weight(s) and bias factor(s). The inputs of each node may be scalar, vectors, matrices, objects, data structures and/or other items or references thereto. Each node may store its respective activation function, weight (if any) and bias factors (if any) independent of other nodes. In some embodiments, the decision of one or more output nodes of the neural network output layer can be calculated or determined using a scoring function and/or decision tree function, using the previously determined weight and bias factors, as is understood in the art.

In some embodiments, the classification (output of the second neural network) can be one or more conclusions as to whether the subject has a neurodegenerative pathology, or a precursor to a neurodegenerative pathology, or is pre-screened for potential of neurodegenerative pathology and requires further investigation. Such neurodegenerative pathology conclusions can be based on one or a plurality of pathologies that are classified by the neural network, and determined or calculated using e.g., a combined weighted score, scorecard, or probabilistic determination. For example, the presence or probabilistic classification of both Amyloid Beta and Tau neurofibrillary tangles may lead to a higher probability conclusion of a neurodegenerative pathology. In some embodiments, the conclusions can also be based on the changes over time of the patient physiology, for example by comparing with previous polarimetric or spectroscopy information of the patient. In some embodiments, the hyperspectral polarimetric or reflectance information is also used as input information to the neural network, which further assists in classifying neurodegenerative pathologies.

From the foregoing description, it will be apparent that variations and modifications may be made to the embodiments of the present disclosure to adopt it to various usages and conditions. Such embodiments are also within the scope of the following claims.

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. An imaging system comprising:
a light source configured to emit light to illuminate a retina of an eye with the light;
one or more imaging devices configured to receive light from the retina via one or more polarizers to be used for generating one or more spectropolarimetric images of the retina; and
a computing device configured to receive the one or more spectropolarimetric images of the retina, evaluate the one or more spectropolarimetric images, and identify one or more biomarkers indicative of a pathology.

2. The imaging system of claim 1, wherein the one or more imaging devices comprises a snapshot camera or a hyperspectral camera.

3. (canceled)

4. The imaging system of claim 1, wherein the one or more polarizers are:
positioned between the one or more imaging devices and the eye, wherein the one or more imaging devices are further configured to receive light from the retina via the one or more polarizers to generate the one or more spectropolarimetric images of the retina from polarized light passing through the one or more polarizers; or
positioned between the light source and the eye, and wherein the light source is further configured to emit the light via the one or more polarizers to illuminate the retina of the eye with polarized light passing through the one or more polarizers.

5. (canceled)

6. The imaging system of claim 1, wherein the light source comprises:
one or more emitters configured to emit light towards one or more dichroic filters,
wherein the one or more dichroic filters are configured to reflect light from the one or more emitters towards the retina of the eye, and
wherein each of the one or more emitters are configured to emit the light through a corresponding dichroic filter of the one or more dichroic filters to illuminate the retina of the eye with the light.

7. The imaging system of claim 1, wherein the light source comprises:
a broadband emitter configured to emit light towards a tunable spectral sampling device, and
wherein the tunable spectral sampling device is configured to filter the light from the broadband emitter towards the retina of the eye to illuminate the retina of the eye with the light.

8. The imaging system of claim 1, wherein the light source comprises:
a broadband emitter configured to emit light towards a filter, and
wherein the filter is configured to filter the light from the broadband emitter towards the retina of the eye to illuminate the retina of the eye with the light.

9. The imaging system of claim 1, wherein the light source comprises a broadband tunable emitter configured to emit light towards the retina of the eye to illuminate the retina of the eye with the light.

10. The imaging system of claim 1, wherein the light source comprises:
a broadband emitter configured to emit light towards a diffractive grating element,
wherein the diffractive grating element is configured to reflect the light from the broadband emitter towards a filter, and
wherein the filter is configured to reflect, based on one or more scanning elements, the light from the diffractive grating element towards of the eye to illuminate the retina of the eye with the light.

11. The imaging system of claim 1, wherein the light source comprises:
a scanning element configured to scan light emissions; and
a broadband emitter configured to emit, based on the scanning element, light towards a prism,
wherein the prism is configured to reflect the light from the broadband emitter towards a spectral filter,
wherein the spectral filter is configured to filter the light from the prism towards an opening, and
wherein the opening is configured to pass the light from the spectral filter towards the eye to illuminate the retina of the eye with the light.

12. The imaging system of claim 1, wherein the one or more imaging devices comprises:
one or more dichroic filters configured to reflect light from the retina of the eye towards one or more light monochromatic sensors, and
wherein the one or more light monochromatic sensors are configured to sense the light from the one or more dichroic filters to generate the one or more spectropolarimetric images of the retina.

13. The imaging system of claim 1, wherein the one or more imaging devices comprises:
a tunable spectral sampling device configured to filter light from the retina of the eye towards a light monochromatic sensor, and
wherein the light monochromatic sensor is configured to sense the light from the tunable spectral sampling device to generate the one or more spectropolarimetric images of the retina.

14. The imaging system of claim 1, wherein the one or more imaging devices comprises:
a filter configured to filter light from the retina of an eye towards a light monochromatic sensor, and
wherein the light monochromatic sensor is configured to sense the light from the filter to generate the one or more spectropolarimetric images of the retina.

15. The imaging system of claim 1, wherein the one or more imaging devices comprises:
an optical element configured to filter light from the retina of the eye towards a dispersive optical element,
wherein the dispersive optical element is configured to filter the light from the optical element towards a light monochromatic sensor, and
wherein the light monochromatic sensor is configured to sense the light from the dispersive optical element to generate the one or more spectropolarimetric images of the retina.

16. The imaging system of claim 1, wherein the one or more imaging devices comprises:
a diffraction grating element configured to reflect light from the retina of the eye towards one or more scanning elements,
wherein the one or more scanning elements reflect the light from the diffraction grating element towards a light monochromatic sensor, and
wherein the light monochromatic sensor is configured to sense the light from the one or more scanning elements to generate the one or more spectropolarimetric images of the retina.

17. The imaging system of claim 1, wherein the one or more imaging devices comprises:
a prism configured to reflect light from the retina of the eye towards an opening,
wherein the opening is configured to filter the light from the prism towards a light monochromatic sensor, and
wherein the light monochromatic sensor is configured to sense the light from the opening to generate the one or more spectropolarimetric images of the retina.

18. The imaging system of claim 1, wherein the one or more imaging devices comprises:
an optical element configured to reflect light from the retina of the eye towards a filter,
wherein the filter is configured to filter the light from the optical element towards a light monochromatic sensor, and
wherein the light monochromatic sensor is configured to sense the light from the filter to generate the one or more spectropolarimetric images of the retina.

19. An imaging system comprising:
a broadband light source configured to emit light at multiple wavelengths to illuminate an object;
an imaging device comprising:
a polarization filter array positioned to receive light reflected from the object and adjust a polarization of the light reflected from the object to generate polarimetric light;
a spectral filter array positioned to receive polarized light and adjust a spectral state of the polarized light to generate spectropolarimetric light;
a sensor positioned to receive the spectropolarimetric light, wherein the sensor is configured to simultaneously capture, from the spectropolarimetric light, one or more spatial components;
one or more spectral components, and one or more polarimetric components associated with the object to generate one or more spectropolarimetric images; and
a computing device configured to receive the one or more spectropolarimetric images of the object, evaluate the one or more spectropolarimetric images, and identify one or more biomarkers indicative of a pathology.

20. The imaging system of claim 19, wherein the spectral filter array further comprises:
a spectral sampling optical element positioned to receive or pass spectrally decomposed light from or to the polarization filter array or the sensor to generate the one or more spectropolarimetric images by optical focusing, collimation, refraction, diffraction or shaping.

21. The imaging system of claim 19, wherein the imaging device further comprises a microlens array configured to receive light from the object and pass the light to the polarization filter array.

22. The imaging system of claim 19, wherein the spectral filter array comprises spectral dispersing element attached to the polarization filter array and positioned to receive the polarized light and disperse the polarized light by wavelength as it passes to the sensor.

23. (canceled)

24. The imaging system of claim 19, wherein the polarization filter array includes a plurality of polarization pixels that each correspond to a unique polarization angle.

25. The imaging system of claim 19, wherein the imaging device further comprises a microlens array that houses the polarization filter array and the spectral filter array.

26. A method comprising:
emitting, by an ocular imaging system, light to illuminate a retina of an eye with the light;
receiving, by the ocular imaging system, light from the retina via one or more polarizers to generate one or more spectropolarimetric images of the retina; and
evaluating, by the ocular imaging system, the one or more spectropolarimetric images, and
identifying, by the ocular imaging system, one or more biomarkers indicative of a pathology.

27.-41. (canceled)

42. A method comprising:
emitting, by an ocular imaging system, light at multiple wavelengths to illuminate an object;
receiving, by the ocular imaging system, light via a polarization filter array positioned to receive light through a reflected from the object and adjust a polarization of the light reflected from the object to generate polarimetric light;
receiving, by the ocular imaging system, polarized light via a spectral filter array positioned to receive the polarized light and adjust a spectral state of the polarized light to generate spectropolarimetric light;
sensing, by the ocular imaging system, the spectropolarimetric light via a sensor positioned to receive the spectropolarimetric light;
extracting, by the ocular imaging system, from the spectropolarimetric light, one or more spatial components, one or more spectral components, and one or more polarimetric components associated with the object to generate one or more spectropolarimetric images;
receiving, by the ocular imaging system, the one or more spectropolarimetric images of the object;
evaluating, by the ocular imaging system, the one or more spectropolarimetric images; and
identifying, by the ocular imaging system, one or more biomarkers indicative of a pathology.
